(19) United States
(12) Patent Application Publication
(10) Pub. No.: US 2026/0053804 A1
(43) Pub. Date: Feb. 26, 2026

(54) Compositions and Methods for the Restoration of Ciliary Function

(71) Applicant: Yale University, New Haven, CT (US)

(72) Inventors: David Breslow, New Haven, CT (US); Shane Elliott, New Haven, CT (US); Paul Ready, New Haven, CT (US)

(21) Appl. No.: 18/935,000

(22) Filed: Nov. 1, 2024

Related U.S. Application Data
(60) Provisional application No. 63/595,402, filed on Nov. 2, 2023.

(51) Int. Cl. A61K 31/519; A61K 31/4035; A61K 31/4166; A61K 31/443; A61K 31/4741; A61K 45/06; A61P 11/00

(52) U.S. Cl. CPC A61K 31/519; A61K 31/4035; A61K 31/4166; A61K 31/443; A61K 31/4741; A61K 45/06; A61P 11/00

(57) ABSTRACT

Provided are compositions and methods for preventing cilia degeneration and treating diseases and disorders associated therewith.

### CROSS-REFERENCE TO RELATED APPLICATIONS

This application claims the benefit of the U.S. Provisional Application No. 63/595,402, filed Nov. 2, 2023, the disclosure of which is incorporated herein by reference in its entirety.

### STATEMENT OF GOVERNMENT SUPPORT

This invention was made with government support under R35GM137956 awarded by the National Institutes of Health. The government has certain rights in the invention.

### BACKGROUND OF THE INVENTION

The primary cilium is a protrusion from the cell surface that serves as an essential organizing center for diverse signaling pathways (Mill, P., et al., 2023, Nat Rev Genet, 24: 421-441). Formation of primary cilia is a complex multi-step process in which axonemal microtubules extend from the mother centriole and a surrounding ciliary membrane becomes compartmentalized from the adjacent plasma membrane (Breslow, D. K. & Holland, A. J., 2019, Annu Rev Biochem, 88: 691-724; Nachury, M. V. & Mick, D. U., 2019, Nat Rev Mol Cell Biol, 20: 389-405; Zhao, H., et al., 2023, Semin Cell Dev Biol, 133: 20-31; Sanchez, I. & Dynlacht, B. D., 2016, Nat Cell Biol, 18: 711-717). Many proteins contribute to ciliogenesis, and consistent with the key role of cilia in signaling, mutations that impair the function of these proteins cause disorders collectively known as ciliopathies (Reiter, J. F. & Leroux, M. R., 2-17, Nat Rev Mol Cell Biol, 18: 533-547). These ciliopathies are typically autosomal recessive congenital syndromes and are characterized by brain malformations, retinal degeneration, heart defects, skeletal alterations, obesity, and kidney cysts (Reiter, J. F. & Leroux, M. R., 2-17, Nat Rev Mol Cell Biol, 18: 533-547; Braun, D. A. & Hildenbrandt, F., 2017, Perspect Biol, 9: 208-214).

A fundamental feature of primary cilia is that they are dynamic structures that are assembled and disassembled in response to various stimuli including cell growth and differentiation. For example, it has been known for decades that serum mitogens and the cell cycle are master regulators of cilia dynamics, with cilia formed in quiescent cells and resorbed during cell cycle re-entry (Tucker, R. W., et al., 1979, Cell, 18: 1065-1072). Recently, the key serum component that induces cilia disassembly was found to be lysophosphatidic acid (LPA), which signals through the LPAR1 G-protein coupled receptor (GPCR) (Walia, V., et al., 2019, Dev Cell, 50: 229-246; Hu, H. B., et al., 2021, Nat Commun, 12: 662). However, given the diversity of cell types and contexts in which ciliation is regulated, it is likely that additional stimuli and receptors for cilia disassembly contribute to organismal control of ciliary dynamics. In addition, it remains poorly understood how these signals are relayed within the cell and converge upon the microtubule modifying enzymes and membrane remodeling machineries that enact cilia disassembly. Prior work has implicated calcium as an intracellular mediator of cilia disassembly, as calcium has long been known to induce deflagellation in C. reinhardtii (Quarmby, L. M. & Hartzell, H. C., 1994, J Cell Biol, 124: 807-815). Additionally, calcium chelators can inhibit cilia disassembly in mammalian cells (Plotnikova, O. V., et al., 2012, Cell, 23: 2658-2670; Mirvis, M., et al., 2019, Plos Biol, 17: e3000381). However, the physiologic source of calcium has eluded characterization, and other intracellular signaling molecules are also likely to play important roles in inducing cilia disassembly.

In addition to the limited mechanistic understanding of cilia disassembly, little is known about the consequences of errors in this process and their role in disease. One mechanism is that aberrant activation of cilia disassembly leads to cilia loss and defects in ciliary signaling similar to those seen in canonical ciliopathies. An example of this type of pathology is seen in focal cortical dysplasia (FCD) and other focal malformation of cortical development (FMCD) disorders. These neurodevelopmental diseases are caused by somatic mutations that locally impair cortical neuron patterning and function, often resulting in intractable epilepsy (Iffland, P. H. II & Crino, P. B., 2017, Annu Rev Pathol, 12: 547-571). Activating mutations in MTOR are a common cause of FCD, and recently, such mutations were shown to cause cilia loss in cultured fibroblasts, mouse neurons, and patient cerebral cortex (Iffland, P. H. II & Crino, P. B., 2017, Annu Rev Pathol, 12: 547-571; Lim, J. S., et al., 2015, Nat Med, 21: 395-400; Gerasimenko, A., et al., 2023, Neurobiol Dis, 182: 106144; Nakashima, M., et al., 2015, Ann Neurol, 78: 375-386; Park, S. M., et al., 2018, Neuron, 99: 83-97). Furthermore, manipulations that restored cilia were able to mitigate FCD phenotypes. However, because many cellular processes are regulated by MTOR, and because many other genes are mutated in FMCD disorders, targeted cilia restoration has not been investigated as a method of treating FMCD (Saxton, R. A. & Sabatini, D. M., 2017, Cell, 168: 960-976; Chung, C., et al., 2023, Nat Genet, 55: 209-220; Baldassari, S., et al., 2019, Acta Neuropathol, 138: 885-900).

As such, there is a pressing need for compositions and methods that target cilia restoration for the treatment of ciliopathies such as FMCD. The present invention meets this long felt, but unmet, need.

### SUMMARY OF THE INVENTION

In various embodiments, the disclosure provides methods of treating or preventing a ciliopathy in a subject in need thereof comprising administering to the subject a composition comprising at least one inhibitor of at least one protein involved in cilia disassembly. In some embodiments, the ciliopathy is a focal malformation of cortical development (FMCD) disorder. In some embodiments, the FMCD disorder is focal cortical dysplasia (FCD).

In some embodiments, the at least one protein involved in cilia disassembly is selected from the group consisting of coagulation factor II receptor (F2R), sterile alpha and TIR motif containing 1 (SARM1), ryanodine receptor 1 (RyR1), ryanodine receptor 2 (RyR2), ryanodine receptor 3 (RyR3), Ras homolog member A (RhoA), Rho-associated protein kinase 1 (ROCK1), and Rho-associated protein kinase 2 (ROCK2).

In some embodiments, the at least one inhibitor of F2R is at least one selected from the group consisting of vorapaxar, atopaxar, SCH-79797, analogs, derivatives, and prodrugs thereof, and pharmaceutically acceptable salts and hydrates thereof.

In some embodiments, the at least one inhibitor of SARM1 is at least one selected from the group consisting of DSRM-3716, dehydronitrosonisodipine (dHNN), MY-9B, WX-02-37, EV-99, WX-02-34, WX-02-35, 3-oxo-5-((2-(trifulromethyl)phenyl)amino)-2,3-dihydroisothiazole-4-carbonitrile, 5-((2-chlorophenyl)amino)-3-oxo-2,3-dihydroisothiazole-4-carbonitrile, 5-((2-methylsulfonyl)phenyl)amino)-3-oxo-2,3-dihydroisothiazole-4-carbonitrile, 5-((6-chloroquinolin-5-yl)amino)-2-methyl-3-oxo-2,3-dihydroisothiazole-4-carbonitrile, 5-((6-methoxy-2-methylquinolin-5-yl)amino)-2-methyl-3-oxo-2,3-dihydroisothiazole-4-carbonitrile, nicotinic acid mononucleotide (NMN), 2-(4-Pyridinyl)-N-(2,2,2-trifluoroethyl)-1-pyrrolidinecarboxamide, 1-(Pyridin-4-ylmethyl)-3-(2,2,2-trifluoroethyl)urea, (S)-1-Methyl-1-(1-(pyridin-4-yl)ethyl)-3-(2,2,2-trifluoroethyl)urea, (R)-1-methyl-1-(1-(pyridin-4-yl)ethyl)-3-(2,2,2-trifluoroethyl)urea, (S)-1-methyl-1-(1-phenylethyl)-3-(2,2,2-trifluoroethyl)urea, 3-(3-chlorophenyl)-N-(4-methyl-3-(pyridin-4-yl)-1H-pyrazol-5-yl)propenamide, 3-(4-Chlorophenyl)-N-(4-methyl-3-(pyridin-4-yl)-1H-pyrazol-5-yl)propenamide, analogs, derivatives, and prodrugs thereof, and pharmaceutically acceptable salts and hydrates thereof.

In some embodiments, the at least one inhibitor of RyR1 is selected from the group consisting of dantrolene, 6,7-(methylenedioxy)-1-octyl-4-quinolone-3-carboxylic acid, analogs, derivatives, and prodrugs thereof, and pharmaceutically acceptable salts and hydrates thereof.

In some embodiments, the at least one inhibitor of RyR2 is selected from the group consisting of dantrolene, analogs, derivatives, and prodrugs thereof, and pharmaceutically acceptable salts and hydrates thereof.

In some embodiments, the at least one inhibitor of RyR3 is selected from the group consisting of dantrolene, analogs, derivatives, and prodrugs thereof, and pharmaceutically acceptable salts and hydrates thereof.

In some embodiments, the at least one inhibitor of ROCK1 is selected from the group consisting of thiazovivin, Y-27632, DJ4, GSK269962A, WAY-624704, RKI-1447, Chroman 1, azaindole 1, ATI13148, GSK429286A, Y-39983, ripasudil, hydroxyfasudil, netarsudil, ZINC00881524, analogs, derivatives, and prodrugs thereof, and pharmaceutically acceptable salts and hydrates thereof.

In some embodiments, the at least one inhibitor of ROCK2 is selected from the group consisting of thiazovivin, Y-27632, DJ4, GSK269962A, WAY-624704, RKI-1447, Chroman 1, azaindole 1, ATI13148, GSK429286A, Y-39983, ripasudil, fasudil, hydroxyfasudil, netarsudil, ZINC00881524, H-1152, belumosudil, analogs, derivatives, and prodrugs thereof, and pharmaceutically acceptable salts and hydrates thereof.

In some embodiments, the method further comprises administering to the subject at least one inhibitor of lysophosphatidic acid receptor 1 (LPAR1). In some embodiments, the method further comprises administering to the subject at least one inhibitor of mammalian target of rapamycin complex 1 (mTORC1). In some embodiments, the method further comprises administering to the subject at least one Ca²⁺ chelator. In some embodiments, the at least one Ca²⁺ chelator is at least one selected from the group consisting of 1,2-bis(2-aminophenoxy)ethane-N, N,N′,N′-tetraacetic acid tetrakis(acetoxymethyl ester) (BAPTA-AM), ethylenediaminetetraacetic acid (EDTA), ethyleneglycoltetraacetic acid (EGTA), diethylenetriaminepentaacetate (DTPA), hydroxyethylethylenediaminetriacetic acid (HEEDTA), diaminocyclohexanetetraacetic acid (CDRA), 1,2-bis(2-aminophenoxy)ethane-N,N,N′, N′-tetraacetic acid (BAPTA), analogs, derivatives, and prodrugs thereof, and pharmaceutically acceptable salts and hydrates thereof.

In one aspect, the disclosure provides a method of inhibiting cilia disassembly in a cell comprising contacting the cell with at least one inhibitor of at least one protein selected from the group consisting of F2R, SARM1, RyR1, RyR2, RyR3, RhoA, ROCK1, and ROCK2.

In some embodiments, the at least one inhibitor of F2R is at least one selected from the group consisting of vorapaxar, atopaxar, SCH-79797, analogs, derivatives, and prodrugs thereof, and pharmaceutically acceptable salts and hydrates thereof.

In some embodiments, the at least one inhibitor of SARM1 is at least one selected from the group consisting of DSRM-3716, dehydronitrosonisodipine (dHNN), MY-9B, WX-02-37, EV-99, WX-02-34, WX-02-35, 3-oxo-5-((2-(trifulromethyl)phenyl)amino)-2,3-dihydroisothiazole-4-carbonitrile, 5-((2-chlorophenyl)amino)-3-oxo-2,3-dihydroisothiazole-4-carbonitrile, 5-((2-methylsulfonyl)phenyl)amino)-3-oxo-2,3-dihydroisothiazole-4-carbonitrile, 5-((6-chloroquinolin-5-yl)amino)-2-methyl-3-oxo-2,3-dihydroisothiazole-4-carbonitrile, 5-((6-methoxy-2-methylquinolin-5-yl)amino)-2-methyl-3-oxo-2,3-dihydroisothiazole-4-carbonitrile, nicotinic acid mononucleotide (NMN), 2-(4-Pyridinyl)-N-(2,2,2-trifluoroethyl)-1-pyrrolidinecarboxamide, 1-(Pyridin-4-ylmethyl)-3-(2,2,2-trifluoroethyl)urea, (S)-1-Methyl-1-(1-(pyridin-4- yl)ethyl)-3-(2,2,2-trifluoroethyl)urea, (R)-1-methyl-1-(1-(pyridin-4-yl)ethyl)-3-(2,2,2-trifluoroethyl)urea, (S)-1-methyl-1-(1-phenylethyl)-3-(2,2,2-trifluoroethyl)urea, 3-(3-chlorophenyl)-N-(4-methyl-3-(pyridin-4-yl)-1H-pyrazol-5-yl)propenamide, 3-(4-Chlorophenyl)-N-(4-methyl-3-(pyridin-4-yl)-1H-pyrazol-5-yl)propenamide, analogs, derivatives, and prodrugs thereof, and pharmaceutically acceptable salts and hydrates thereof.

In some embodiments, the at least one inhibitor of RyR1 is selected from the group consisting of dantrolene, 6,7-(methylenedioxy)-1-octyl-4-quinolone-3-carboxylic acid, analogs, derivatives, and prodrugs thereof, and pharmaceutically acceptable salts and hydrates thereof.

In some embodiments, the at least one inhibitor of RyR2 is selected from the group consisting of dantrolene, analogs, derivatives, and prodrugs thereof, and pharmaceutically acceptable salts and hydrates thereof.

In some embodiments, the at least one inhibitor of RyR3 is selected from the group consisting of dantrolene, analogs, derivatives, and prodrugs thereof, and pharmaceutically acceptable salts and hydrates thereof.

In some embodiments, the at least one inhibitor of ROCK1 is selected from the group consisting of thiazovivin, Y-27632, DJ4, GSK269962A, WAY-624704, RKI-1447, Chroman 1, azaindole 1, ATI13148, GSK429286A, Y-39983, ripasudil, hydroxyfasudil, netarsudil, ZINC00881524,, analogs, derivatives, and prodrugs thereof, and pharmaceutically acceptable salts and hydrates thereof.

In some embodiments, the at least one inhibitor of ROCK2 is selected from the group consisting of thiazovivin, Y-27632, DJ4, GSK269962A, WAY-624704, RKI-1447, Chroman 1, azaindole 1, ATI13148, GSK429286A, Y-39983, ripasudil, fasudil, hydroxyfasudil, netarsudil, ZINC00881524, H-1152, belumosudil, analogs, derivatives, and prodrugs thereof, and pharmaceutically acceptable salts and hydrates thereof.

In some embodiments, the method further comprises contacting the cell with at least one inhibitor of LPAR1. In some embodiments, the method further comprises contacting the cell with at least one inhibitor of mTORC1. In some embodiments, the method further comprises contacting the cell with at least one Ca²⁺ chelator. In some embodiments, the at least one Ca²⁺ chelator is at least one selected from the group consisting of 1,2-bis(2-aminophenoxy)ethane-N, N,N′,N′-tetraacetic acid tetrakis(acetoxymethyl ester) (BAPTA-AM), ethylenediaminetetraacetic acid (EDTA), ethyleneglycoltetraacetic acid (EGTA), diethylenetriaminepentaacetate (DTPA), hydroxyethylethylenediaminetriacetic acid (HEEDTA), diaminocyclohexanetetraacetic acid (CDRA), 1,2-bis(2-aminophenoxy)ethane-N,N,N′,N′-tetraacetic acid (BAPTA), analogs, derivatives, and prodrugs thereof, and pharmaceutically acceptable salts and hydrates thereof.

In various embodiments, the disclosure provides compositions comprising: a) at least one inhibitor of at least one protein selected from the group consisting of F2R, SARM1, RyR1, RyR2, RyR3, RhoA, ROCK1, and ROCK2; and b) at least one selected from the group consisting of: (i) at least one inhibitor of LPAR1; (ii) at least one inhibitor of mTORC1; and (iii) at least one Ca²⁺ chelator. In some embodiments, the at least one inhibitor of at least one protein selected from the group consisting of F2R, SARM1, RyR1, RyR2, RyR3, RhoA, ROCK1, and ROCK2 is at least one selected from the group consisting of vorapaxar, atopaxar, SCH-79797, argatroban, bivalirudin, dabigatran, lepirudin, desirudin, dantrolene, 6,7-(methylenedioxy)-1-octyl-4-quinolone-3-carboxylic acid, NSC 23766, zoledronic acid, EHT 1864, CCF-1423, ML141, thiazovivin, Y-27632, DJ4, GSK269962A, WAY-624704, RKI-1447, Chroman 1, azaindole 1, ATI13148, GSK429286A, Y-39983, ripasudil, hydroxyfasudil, netarsudil, ZINC00881524, H-1152, belumosudil, DSRM-3716, dehydronitrosonisodipine (dHNN), MY-9B, WX-02-37, EV-99, WX-02-34, WX-02-35, 3-oxo-5-((2-(trifulromethyl)phenyl)amino)-2,3-dihydroisothiazole-4-carbonitrile, 5-((2-chlorophenyl)amino)-3-oxo-2,3-dihydroisothiazole-4-carbonitrile, 5-((2-methylsulfonyl)phenyl)amino)-3-oxo-2,3-dihydroisothiazole-4-carbonitrile, 5-((6-chloroquinolin-5-y1)amino)-2-methyl-3-oxo-2,3-dihydroisothiazole-4-carbonitrile, 5-((6-methoxy-2-methylquinolin-5-yl)amino)-2-methyl-3-oxo-2,3-dihydroisothiazole-4-carbonitrile, nicotinic acid mononucleotide (NMN), 2-(4-Pyridinyl)-N-(2,2,2-trifluoroethyl)-1-pyrrolidinecarboxamide, 1-(Pyridin-4-ylmethyl)-3-(2,2,2-trifluoroethyl)urea, (S)-1-Methyl-1-(1-(pyridin-4- yl)ethyl)-3-(2,2,2-trifluoroethyl)urea, (R)-1-methyl-1-(1-(pyridin-4-yl)ethyl)-3-(2,2,2-trifluoroethyl)urea, (S)-1-methyl-1-(1-phenylethyl)-3-(2,2,2-trifluoroethyl)urea, 3-(3-chlorophenyl)-N-(4-methyl-3-(pyridin-4-yl)-1H-pyrazol-5-yl)propenamide, 3-(4-Chlorophenyl)-N-(4-methyl-3-(pyridin-4-yl)-1H-pyrazol-5-yl)propenamide, analogs, derivatives, and prodrugs thereof, and pharmaceutically acceptable salts and hydrates thereof. In some embodiments, the at least one Ca²⁺ chelator is at least one selected from the group consisting of 1,2-bis(2-aminophenoxy)ethane-N, N,N′,N′-tetraacetic acid tetrakis(acetoxymethyl ester) (BAPTA-AM), ethylenediaminetetraacetic acid (EDTA), ethyleneglycoltetraacetic acid (EGTA), diethylenetriaminepentaacetate (DTPA), hydroxyethylethylenediaminetriacetic acid (HEEDTA), diaminocyclohexanetetraacetic acid (CDRA), 1,2-bis(2-aminophenoxy)ethane-N,N,N′,N′-tetraacetic acid (BAPTA), analogs, derivatives, and prodrugs thereof, and pharmaceutically acceptable salts and hydrates thereof.

### BRIEF DESCRIPTION OF THE DRAWINGS

The following detailed description of exemplary embodiments of the invention will be better understood when read in conjunction with the appended drawings. For the purpose of illustrating the invention, there are shown in the drawings exemplary embodiments. It should be understood, however, that the invention is not limited to the precise arrangements and instrumentalities of the embodiments shown in the drawings.

FIG. 1, comprising FIG. 1A through FIG. 1F, depicts representative results of a CRISPRa screen to identify negative regulators of cilia dynamics. FIG. 1A depicts a schematic representation of the Hh pathway including key genes and activators. Pathway activation is triggered by Shh-mediated derepression of Smo or can be induced by Smo agonist SAG. Smo then initiates the cilia-dependent activation of Gli family transcription factors. A synthetic reporter gene containing Gli binding sites (8× Gli-BS) can convert Hh pathway activity into expression of a blasticidin resistance gene (BlastR). FIG. 1B depicts representative Blastidicin resistance assessed in wildtype NIH-3T3 cells with Shh-BlastR reporter and dCas9-VP64-BFP following Hh pathway stimulation (250 nM SAG, 24 hr) or in unstimulated cells (-SAG). FIG. 1C depicts a schematic representation of the pooled CRISPRa screening strategy. Representative cells receiving an sgRNA that reduces ciliation (purple), an sgRNA that impairs ciliary Hh signaling (blue), and a negative control or non-hit sgRNA (gray) are shown. FIG. 1D depicts a representative volcano plot of genome-wide CRISPRa screen results showing casTLE P values versus effect size, with Gli family genes and select hits highlighted. FIG. 1E depicts representative changes in normalized sgRNA abundance in blasticidin-selected versus unselected cell pools were determined by deep sequencing and plotted for the indicated sets of sgRNAs.

FIG. 1F depicts representative expression of endogenous Hh target gene Glil was assessed by quantitative RT-PCR for parental NIH-3T3 cells and for cells transduced with sgRNAs targeting the indicated genes, with and without SAG treatment. Values shown are normalized to the level of Glil expression in unstimulated wildtype cells. Bars show means of N≥3 independent experiments (circles).

FIG. 2, comprising FIG. 2A through FIG. 2G, depicts representative results demonstrating that overexpression of F2R or SARM1 induces cilia disassembly. FIG. 2A depicts a schematic diagram of the GPCR F2R highlighting its activation via N-terminal proteolytic cleavage, inhibition by the drug Vorapaxar, and signaling through G proteins. FIG. 2B depicts representative images of parental NIH-3T3 cells and cells transduced with F2R CRISPRa sgRNAs were serum starved for 24 hr, followed by immunofluorescence analysis of cilia (marked by Arl13b) and centrioles (marked by g-tubulin; g-tub). Where indicated, cells were treated with 6 μM Vorapaxar during serum starvation. Scale bar: 5 μm. FIG. 2C depicts representative immunofluorescence analysis of ciliation in NIH-3T3 cells expressing a Doxycycline (Dox)-inducible F2R transgene (NIH-3T3 Tet-F2R-Flag). Cells were treated as indicated with 1 μg/ml Dox and Vorapaxar during 24 hr serum starvation. Bars show means of N=3 independent experiments (circles; n>125 cells each); error bars show standard deviation. Asterisks denote significant (*, P<0.05; **, P<0.01; ***, P<0.001; ****, P<0.0001) differences in mean. FIG. 2D depicts representative immunofluorescence analysis of ciliation in NIH-3T3 Tet-F2R-Flag cells. Cells were serum starved for 24 hr, and then either left untreated or treated with Dox for a further 24 hours. Lines show mean and standard deviation from N=3 independent experiments (n>95 cells analyzed per replicate/condition). FIG. 2E depicts a representative diagram of SARM1 highlighting regulatory ARM domains, octamer-forming SAM domains, and NADase TIR domain that cleaves NAD+ to produce nicotinamide (NAM) and cyclic-ADP-ribose (cADPR). Small molecule inhibitor DSRM-3716 is shown. FIG. 2F depicts representative images of parental NIH-3T3 cells and cells transduced with SARM1 CRISPRa sgRNAs were serum starved for 24 hr, followed by immunofluorescence analysis of cilia (marked by Arl13b) and centrioles (marked by g-tub). Where indicated, cells were treated with 30 μM DSRM-3716 during serum starvation. Scale bar: 5 μm. FIG. 2G depicts a representative quantification of ciliation as analyzed in FIG. 2F. Bars show means of N=3 independent replicates (circles; n>80 cells each); error bars show standard deviation. Asterisks denote significant differences in mean as in FIG. 2C.

FIG. 3, comprising FIG. 3A through FIG. 3E, depicts representative results demonstrating that a SARM1-triggered signaling pathway mediates cilia disassembly. FIG. 3A depicts a schematic representation of cilia disassembly pathway in which F2R triggers SARM1-mediated cADPR production and stimulation of Ryanodine Receptor (RyR)-mediated calcium release from the endoplasmic reticulum. SARM1 inhibitors DSRM-3716 and dHNN (dehydronitrosonisodipine), RyR inhibitor dantrolene, and calcium chelator BAPTA-AM are also shown. FIG. 3B depicts representative immunofluorescence analysis of ciliation in NIH-3T3 Tet-F2R-Flag cells following treatment with 1 μg/ml Dox, DSRM-3716, and/or 10 μM dHNN as indicated during 24 hr serum starvation. Bars show means of N≥3 independent experiments (circles; n>100 cells each); error bars show standard deviation. Asterisks denote significant (*, P<0.05; **, P<0.01; *, P<0.001; **, P<0.0001) differences in mean. FIG. 3C depicts representative immunofluorescence analysis of ciliation in parental NIH-3T3 cells and cells transduced with SARM1 CRISPRa sgRNAs. Where indicated, cells were treated with 10 μM dantrolene for 24 hr followed by serum starvation for 24 hr in the continued presence of dantrolene. Bars show means of N=3 independent replicates (circles, each representing n>80); error bars show standard deviation. Asterisks denote significant differences in mean (as in FIG. 3B). FIG. 3D depicts representative images of NIH-3T3 Tet-F2R-Flag cells were serum starved for 24 hr, followed by immunofluorescence analysis of cilia (marked by Arl13b) and centrioles (marked by g-tubulin; g-tub). Where indicated, cells were treated with 1 μg/ml Dox, 5 μM dantrolene, 5 μM BAPTA-AM, and/or 10 μM Y-27632 during serum starvation. As in FIG. 3C, dantrolene was added 24 hr prior to serum starvation. Scale bar: 5 μm. FIG. 3E depicts a representative quantification of ciliation in NIH-3T3 Tet-F2R-Flag cells following treatment with Dox, dantrolene, BAPTA, and/or Y-27632 as in FIG. 3B and FIG. 3D. Bars show means of N≥3 independent experiments (circles, each representing n>85); error bars show standard deviation. Asterisks denote significant differences in mean (as in FIG. 3B). FIG. 4, comprising FIG. 4A though FIG. 4G, depicts representative results demonstrating endogenous regulation of cilia dynamics by a SARM1-driven pathway. FIG. 4A depicts representative immunofluorescence analysis of ciliation in NIH-3T3 cells following serum-induced cilia disassembly. After 48 hr serum starvation, serum was added for 24 hr along with 30 μM DSRM-3716, 10 μM dHNN, or 10 μM dantrolene, 10 M Y-27632,or 100 nM Cytochalasin D (CytoD), as indicated. Bars show means of N≥3 independent experiments (circles; n>80 cells each); error bars show standard deviation. Asterisks denote significant (*, P<0.05; **, P<0.01; ***, P<0.001; ****, P<0.0001) differences in mean. FIG. 4B depicts representative analysis of ciliation in RPE1 cells following LPA-induced cilia disassembly. Cells were serum starved for 48 hr then stimulated with 15 M LPA for 48 hr. During LPA stimulation, cells were treated with DSRM-3716, dHNN, dantrolene, Y-27632,or Cytochalasin D (CytoD). Bars show means of N=3 independent experiments (circles; n>80 cells each); error bars show standard deviation. Asterisks denote significant differences in mean (as in FIG. 4A). FIG. 4C depicts representative analysis of ciliation in RPE1 cells following exposure to thrombin. Cells were serum starved for 48 hr prior to addition of 10 U/ml thrombin for 48 hr, with or without Vorapaxar or DSRM-3716. Bars show means of N=3 independent experiments (circles; n>140 cells each); error bars show standard deviation. Asterisks denote significant differences in mean (as in FIG. 4A). FIG. 4D depicts representative analysis of ciliation in serum-starved RPE1 cells after treatment with thrombin. Thin lines show each of N=3 replicates (n>100 cells each), and thick lines show means. Asterisks denote significant differences in mean between untreated and thrombin-treated cells at the corresponding timepoints (as in FIG. 4A). FIG. 4E depicts representative images of thrombin-induced cilia disassembly assessed by live-cell imaging of RPE1 cells expressing ciliary marker Htr6-3xmNeonGreen and centriolar marker miRFP670-Centrin2. The time elapsed after addition of thrombin (10 U/ml) is noted in hrs: min: sec. Scale bar: 5 mm. See also FIG. 9C. FIG. 4F depicts representative quantification of cilia length in cells treated as in FIG. 4B. Violin plots show combined length distributions from N=3 independent replicates [center line=median; dotted lines=25th and 75th percentiles]; circles show means from each replicate (n>50 cilia per replicate). Asterisks denote significant differences in mean. FIG. 4G depicts representative analysis of ciliation in unstarved NIH-3T3 cells treated with DSRM-3716, dHNN, dantrolene, Y-27632, or CytoD for 24 hr. Control-FBS cells were serum starved for 24 hr. Bars show means of N≥3 experiments (circles; n>80 cells each); error bars show standard deviation. Asterisks denote significant differences in mean.

FIG. 5, comprising FIG. 5A through FIG. 5E, depicts representative results demonstrating Cilia disassembly pathway components are mutated in focal malformations of cortical development. FIG. 5A depicts a schematic representation of cilia disassembly pathway (Top). GPCRs Lpar1 and F2R receive signals to stimulate cilia disassembly, leading to SARM1-mediated cADPR production and Ryanodine Receptor (RyR)-mediated release of calcium from the endoplasmic reticulum. RhoA is activated downstream of SARM1 and GPCRs, leading to increased Rock kinase activity. Rock-mediated actomyosin contractility and calcium-dependent activation of Aurora A and Hdac6 induce cilia disassembly. Asterisks denote genes mutated in FMCD. Overlap of cilia disassembly pathway genes and genes mutated in FMCD is shown (Bottom) (P value determined by Fisher's exact test). MTOR negative regulators TSC1 and TSC2 that are biallelically inactivated in FMCD are shown in italics. FIG. 5B depicts representative immunofluorescence analysis of ciliation in NIH-3T3 Tet-SARM1-Flag (WT, G528S, and V331E) cells following treatment with 1 μg/ml Dox, 500 mM 3-Acetylpyridine (3AP), and/or 30 μM DSRM-3716 as indicated during 24 hr serum starvation. Bars show means of N=3 independent experiments (circles; n>115 cells each); error bars show standard deviation. Asterisks denote significant (*, P<0.05; **, P<0.01; ***, P<0.001; ****, P<0.0001) differences in mean. FIG. 5C depicts representative analysis of ciliation in NIH-3T3 Tet-Flag-RhoA cells (P75S and Q63L) following treatment with 1 μg/ml Dox and/or 30 μM Y-27632 as indicated during 24 hr serum starvation. Bars show means of N=3 independent experiments (circles; n>140 cells each); error bars show standard deviation. Asterisks denote significant differences in mean (as in FIG. 5B). FIG. 5B depicts a representative quantification of the length of Arl13b-labeled cilia analyzed in cultured primary cortical neurons, identified by CaMKII staining, after being cultured for 14 days in vitro, followed by 24 hr treatment with DMSO or 30 μM DSRM-3716, as indicated. Centrioles are marked by Fop. Circles show mean cilia length from N=3 replicate experiments (n>30 cilia per experiment). P=0.0005 (Kolmogorov-Smirnov test). FIG. 5E depicts a representative analysis of ciliation in NIH-3T3 wildtype and Tsc2 knockout cells following 24 hr serum starvation and treatment with 250 nM Torin1 or 30 μM DSRM-3716. Bars show means of N=3 independent experiments (circles; n>140 cells each); error bars show standard deviation. Asterisks denote significant differences in mean (as in FIG. 5B).

FIG. 6, comprising FIG. 6A through FIG. 6B, depicts representative results of validation of CRISPRa Shh signaling reporter cell line. FIG. 6A depicts representative quantification of relative viability of NIH-3T3 cells with Shh-BlastR reporter and dCas9-VP64-BFP transduced with CRISPRa sgRNAs targeting the indicated genes. Blastidicin resistance was then assessed following Hh pathway stimulation (250 nM SAG, 24 hr) or in unstimulated cells (-SAG). FIG. 6B depicts a representative quantification of frequency of a mixture of wildtype (mCherry-negative) NIH-3T3 Shh-BlastR reporter cells and reporter cells co-expressing a Gli3 CRISPRa sgRNA and mCherry (sgGli3-mCherry) analyzed by flow cytometry. The fraction of mCherry-positive cells is indicated after 24 hr SAG-mediated Hh pathway activation followed by no treatment (unselected) or treatment with blastidicin for 4 d.

FIG. 7, comprising FIG. 7A through FIG. 7H, depicts representative results demonstrating overexpression of F2R or SARM1 reduces Hh signaling and ciliation. FIG. 7A depicts a representative quantification of the expression of Hh target gene Glil assessed by quantitative RT-PCR for parental NIH-3T3 cells and for cells transduced with sgRNA F2R. B, with and without SAG treatment. Bars show means of N=3 independent experiments (circles). FIG. 7B depicts a representative quantification of the expression of F2R assessed for parental NIH-3T3 cells and for cells transduced with F2R CRISPRa sgRNAs as in FIG. 7A. Bars show means of N=3 independent experiments (circles). FIG. 7C depicts representative images of parental NIH-3T3 cells and cells transduced with F2R CRISPRa sgRNAs were serum starved for 24 hr, followed by immunofluorescence analysis of cilia (marked by acetylated tubulin; AcTub) and mother centrioles (marked by Cep164). Where indicated, cells were treated with 6 μM Vorapaxar during serum starvation. Scale bar: 5 μm. FIG. 7D depicts representative parental NIH-3T3 cells and cells transduced with a Brs3 CRISPRa sgRNA were serum starved for 24 hr, followed by immunofluorescence analysis of cilia (marked by Arl13b) and mother centrioles (marked by Cep164). Scale bar: 5 μm. FIG. 7E depicts representative immunofluorescence analysis of ciliation in RPE1 cells with Tet-F2R-Flag transgene. During 48 hr serum starvation, cells were treated with 1 μg/ml doxycycline (Dox) and 2 μM tubacin as indicated. Bars show means of N=3 independent experiments (circles; n>150 cells each); error bars show standard deviation. Asterisks denote significant (*, P<0.05; **, P<0.01; ***, P<0.001; ****, P<0.0001) differences in mean. FIG. 7F depicts a representative quantification of expression of Hh target gene Glil assessed by quantitative RT-PCR for parental NIH-3T3 cells and for cells transduced with sgRNA SARM1.B, with and without SAG treatment. Bars show means of N=3 independent experiments (circles). FIG. 7G depicts a representative quantification of expression of SARM1 assessed for parental NIH-3T3 cells and for cells transduced with SARM1 CRISPRa sgRNAs as in FIG. 7F. Bars show means of N=3 independent experiments (circles). FIG. 7H depicts representative images of parental NIH-3T3 cells and cells transduced with SARM1 CRISPRa sgRNAs were serum starved for 24 hr, followed by immunofluorescence analysis of cilia (marked by acetylated tubulin; AcTub) and mother centrioles (marked by Cep164). Where indicated, cells were treated with 30 μM DSRM-3716 during serum starvation. Scale bar: 5 μm.

FIG. 8 depicts representative results demonstrating Rock kinase inhibition blocks cilia loss in SARM1-overexpressing cells. Immunofluorescence analysis of ciliation in parental NIH-3T3 cells and cells transduced with SARM1 CRISPRa sgRNAs after serum starvation for 24 hr. Where indicated, cells were treated with 10 μM Y-27632 during serum starvation. Bars show means of N=3 independent replicates (circles; n>80 cells each); error bars show standard deviation. Asterisks denote significant (*, P<0.05; **, P<0.01; ***, P<0.001; ****, P<0.0001) differences in mean.

FIG. 9, comprising FIG. 9A through FIG. 9E, depicts representative results demonstrating that SARM1-directed pathway is a key endogenous regulator of cilia dynamics. FIG. 9A depicts representative immunofluorescence analysis of ciliation in NIH-3T3 cells following serum-induced cilia disassembly. After serum starvation for 48 hr, serum was added for 24 hr with or without 6 μM Vorapaxar, as indicated. Bars show means of N≥3 independent experiments (circles; n>80 cells each); error bars show standard deviation. NS (not significant; P>0.05 for difference in mean assessed). FIG. 9B depicts a representative analysis of cilia length in serum-starved RPE1 cells treated with 10 U/ml thrombin as indicated. Lines show means from n>100 cilia across N=3 replicate experiments. Asterisks denote significant (*, P<0.05; **, P<0.01; ***, P<0.001; ****, P<0.0001) differences in mean (Kolmogorov-Smirnov test). FIG. 9C depicts representative images of thrombin-triggered cilia disassembly assessed by live-cell imaging of RPE1 cells expressing ciliary marker (Htr6-3xmNeonGreen) and centriole marker (miRFP670-Centrin2). The time after thrombin addition (10 U/ml) is noted in hrs: min: sec. Scale bar: 5 μm. FIG. 9D depicts representative immunofluorescence analysis of cilia (marked by Arl13b) and centrioles (marked by γ-tubulin; γ-tub) in NIH-3T3 cells that were serum starved for 48 hr then stimulated with 15 μM LPA for 48 hr along with indicated inhibitors (as in FIG. 5B-C). -LPA cells were not treated with LPA and grown in serum starvation medium for 96 hr. Scale bar: 5 μm. FIG. 9E depicts representative immunofluorescence analysis of ciliation in unstarved RPE1 cells treated with 30 μM DSRM-3716, 10 μM dHNN, or 10 μM dantrolene, 10 μM Y-27632,or 100 nM Cytochalasin D (CytoD) for 48 hr. Control-FBS cells were serum starved for 48 hr. Bars show means of N=3 independent experiments (circles; n>90 cells each); error bars show standard deviation.

Asterisks denote significant differences in mean as in FIG. 9B.

FIG. 10, comprising FIG. 10 through FIG. 10E, depicts representative results demonstrating roles for cilia disassembly pathway components in focal malformations of cortical development. FIG. 10A depicts representative immunofluorescence analysis of ciliation in NIH-3T3 Tet-SARM1-Flag (SARM1 WT, SARM1 G528S, and SARM1 V331E) cells following treatment with 1 μg/ml Dox during 24 hr serum starvation. Bars show means of N=3 independent experiments (circles; n>120 cells each); error bars show standard deviation. FIG. 10B depicts representative quantification of ciliation in excitatory cortical neurons cultured for 14 d in vitro followed by 24 hr treatment with DMSO or 30 μM DSRM-3716 as indicated. Bars show means of N=3 independent experiments (circles; n>30 cells each); error bars show standard deviation. FIG. 10C depicts representative immunofluorescence analysis of phosphorylated ribosomal protein S6 (Phospho-S6; an MTOR pathway marker) is shown for wildtype NIH-3T3 cells and Tsc2 knockout cells. Cells were analyzed after 24 hr serum starvation and treatment with 250 nM Torin1 or 30 μM DSRM-3716 as indicated. Scale bar: 10 μm. FIG. 10D depicts representative immunofluorescence analysis of cilia (Arl13b) and centrioles (γ-tubulin; γ-tub) is shown for wildtype NIH-3T3 cells and Tsc2 knockout cells treated as in c. Scale bar: 10 μm. FIG. 10E depicts a representative schematic of characteristics of different potential types of ciliopathies. Left: normal cilia homeostasis is the result of a balance between cilia disassembly and cilia assembly. Middle: in canonical ciliopathies such as Oral-facial-digital (OFD) syndrome, Joubert syndrome, or Meckel syndrome, loss of function mutations in genes needed for assembly of functional cilia cause disease. Right: in a non-canonical ciliopathy potentially exemplified by FCD, somatic activating mutations in cilia disassembly genes disturb cilia homeostasis and cause cilia loss.

FIG. 11, comprising FIG. 11A through FIG. 11D, depicts representative results demonstrating that SARM1 mediates cilia dynamics in primary cortical neurons. FIG. 11A depicts a schematic representation of the SARM1 pathway. FIG. 11B depicts representative images of primary cortical neurons stained with ARL13b, γ-tubulin, and pS6 with and without DSRM-3716 treatment. FIG. 11C depicts representative images and quantification of cilia length in primary cortical neurons with and without DSRM-3716 treatment. FIG. 11D depicts representative quantification of percent ciliated primary cortical neuron cells treated with the indicated shRNA and inhibitors.

### DETAILED DESCRIPTION OF THE INVENTION

In various embodiments, the disclosure relates to methods for the treatment and/or prevention of ciliopathies, for example treatment of focal malformation of cortical development (FCMD) disorders (e.g., focal cortical dysplasia, FCD), and other disorders in which ciliary dysfunction contributes to pathology (e.g., medulloblastoma). In some embodiments, the methods of treatment comprise restoration of cilia. In some embodiments, the methods comprise administering at least one composition which inhibits at least one protein involved in cilia disassembly. In some embodiments, the at least one protein involved in cilia disassembly is selected from the group consisting of coagulation factor II receptor (F2R), sterile alpha and TIR motif containing 1 (SARM1), ryanodine receptor 1 (RyR1), ryanodine receptor 2 (RyR2), ryanodine receptor 3 (RyR3), Ras homolog member A (RhoA), Rho-associated protein kinase 1 (ROCK1), and Rho-associated protein kinase 2 (ROCK2).

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

The term “activate,” as used herein, means to induce or increase an activity or function, for example, about ten percent relative to a control value. In some embodiments, the activity is induced or increased by 50% compared to a control value. In some embodiments, the activity is induced or increased by 75% compared to a control value. In some embodiments, the activity is induced or increased by 95% compared to a control value. “Activate,” as used herein, also means to increase a molecule, a reaction, an interaction, a gene, an mRNA, and/or a protein's expression, stability, function or activity by a measurable amount or to increase entirely. Activators are compounds that, e.g., bind to, partially or totally induce stimulation, increase, promote, induce activation, activate, sensitize, or up regulate a protein, a gene, and an mRNA stability, expression, function and activity, e.g., agonists.

The phrase “inhibit,” as used herein, means to reduce a molecule, a reaction, an interaction, a gene, an mRNA, and/or a protein's expression, stability, function or activity by a measurable amount or to prevent entirely. Inhibitors are compounds that, e.g., bind to, partially or totally block stimulation, decrease, prevent, delay activation, inactivate, desensitize, or down regulate a protein, a gene, and an mRNA stability, expression, function and activity, e.g., antagonists. In one embodiment, the activity is suppressed or blocked by 10% compared to a control value. In one embodiment, the activity is suppressed or blocked by 50% compared to a control value. In one embodiment, the activity is suppressed or blocked by 75%. In one embodiment, the activity is suppressed or blocked by 95%.

As used herein, the term “analog,” “analogue,” or “derivative” is meant to refer to a chemical compound or molecule made from a parent compound or molecule by at least one chemical reaction. As such, an analog can be a structure having a structure similar to that of the small molecule therapeutic agents described herein or can be based on a scaffold of a small molecule therapeutic agents described herein, but differing from it in respect to certain components or structural makeup, which may have a similar or opposite action metabolically. An analog or derivative can also be a small molecule that differs in structure from the reference molecule, but retains the essential properties of the reference molecule. An analog or derivative may change its interaction with certain other molecules relative to the reference molecule. An analog or derivative molecule may also include a salt, an adduct, tautomer, isomer, or other variant of the reference molecule.

The term “tautomers” refers to constitutional isomers of organic compounds that readily interconvert by a chemical process (tautomerization).

The term “isomers” or “stereoisomers” refer to compounds, which have identical chemical constitution, but differ with regard to the arrangement of the atoms or groups in space.

The term “prodrug” refers to compounds that differ in structure from the reference molecule, but is chemically modified by a particular cellular process to ultimately become modified to retain the essential properties of the reference molecule or become the reference molecule.

The term “dysregulation” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the normally “regulated” (expected) respective characteristic. Characteristics which are regulated or expected for one cell or tissue type, might be dysregulated for a different cell or tissue type.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of at least one sign or symptom of the disease or disorder, the frequency with which such at least one sign or symptom is experienced by a patient, or both, is reduced.

The terms “patient,” “subject,” “individual,” and the like are used interchangeably herein, and refer to any animal, or cells thereof whether in vitro or in situ, amenable to the methods described herein. In some embodiments, the patient, subject or individual is a mammal such as a non-primate (e.g., cows, pigs, horses, cats, dogs, rats, etc.) and a primate (e.g., monkey and human). In some embodiments, the patient, subject or individual is a human.

As used herein, the term “pharmaceutical composition” refers to a mixture of at least one compound useful within the invention with a pharmaceutically acceptable carrier. The pharmaceutical composition facilitates administration of the compound to a patient or subject.

Multiple techniques of administering a compound exist in the art including, but not limited to, intravenous, oral, aerosol, parenteral, ophthalmic, pulmonary, and topical administration.

A “therapeutic” treatment is a treatment administered to a subject who exhibits signs or symptoms of pathology disease or disorder, for the purpose of diminishing or eliminating those signs or symptoms.

As used herein, the term “treatment” or “treating” is defined as the application or administration of a therapeutic agent, i.e., a compound of the invention (alone or in combination with another pharmaceutical agent), to a patient, or application or administration of a therapeutic agent to an isolated tissue or cell from a patient (e.g., for diagnosis or ex vivo applications), who has a disease or disorder contemplated herein, a sign or symptom of a disease or disorder contemplated herein or the potential to develop a disease or disorder contemplated herein, with the purpose to cure, heal, alleviate, relieve, alter, remedy, ameliorate, improve or affect a disease or disorder contemplated herein, the signs or symptoms of a disease or disorder contemplated herein or the potential to develop a disease or disorder contemplated herein. Such treatments may be specifically tailored or modified, based on knowledge obtained from the field of pharmacogenomics.

As used herein, the terms “effective amount,” “pharmaceutically effective amount” and “therapeutically effective amount” refer to a sufficient amount of an agent to provide the desired biological or physiologic result. That result may be reduction and/or alleviation of at least one sign, symptom, or a cause of a disease or disorder, or any other desired alteration of a biological system. An appropriate effective amount in any individual case may be determined by one of ordinary skill in the art using routine experimentation.

As used herein, the term “pharmaceutically acceptable” refers to a material, such as a carrier or diluent, which does not abrogate the biological activity or properties of the compound, and is relatively non-toxic, i.e., the material may be administered to an individual without causing an undesirable biological effect or interacting in a deleterious manner with any of the components of the composition in which it is contained.

As used herein, the language “pharmaceutically acceptable salt” refers to a salt of the administered compound prepared from pharmaceutically acceptable non-toxic acids, including inorganic acids, organic acids, solvates, hydrates, or clathrates thereof. Examples of such inorganic acids are hydrochloric, hydrobromic, hydroiodic, nitric, sulfuric, phosphoric, acetic, hexafluorophosphoric, citric, gluconic, benzoic, propionic, butyric, sulfosalicylic, maleic, lauric, malic, fumaric, succinic, tartaric, amsonic, pamoic, p-tolunenesulfonic, and mesylic. Appropriate organic acids may be selected, for example, from aliphatic, aromatic, carboxylic and sulfonic classes of organic acids, examples of which are formic, acetic, propionic, succinic, camphorsulfonic, citric, fumaric, gluconic, isethionic, lactic, malic, mucic, tartaric, para-toluenesulfonic, glycolic, glucuronic, maleic, furoic, glutamic, benzoic, anthranilic, salicylic, phenylacetic, mandelic, embonic (pamoic), methanesulfonic, ethanesulfonic, pantothenic, benzenesulfonic (besylate), stearic, sulfanilic, alginic, galacturonic, and the like. Furthermore, pharmaceutically acceptable salts include, by way of non-limiting example, alkaline earth metal salts (e.g., calcium or magnesium), alkali metal salts (e.g., sodium-dependent or potassium), and ammonium salts.

As used herein, the term “pharmaceutically acceptable carrier” means a pharmaceutically acceptable material, composition, or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention and are physiologically acceptable to the patient. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, PA), which is incorporated herein by reference.

As used herein, the term “potency” refers to the dose needed to produce half the maximal response (ED₅₀).

As used herein, the term “efficacy” refers to the maximal effect (Eₘₐₓ) achieved within an assay.

“Measuring” or “measurement,” or alternatively “detecting” or “detection,” means assessing the presence, absence, quantity or amount (which can be an effective amount) of either a given substance within a sample, including the derivation of qualitative or quantitative concentration levels of such substances, or otherwise evaluating the values or categorization of the substance or the sample.

As used herein, “associated” refers to coincidence with the development or manifestation of a disease, condition, or phenotype. Association may be due to, but is not limited to, genes responsible for housekeeping functions, those that are part of a pathway that is involved in a specific disease, condition, or phenotype and those that indirectly contribute to the manifestation of a disease, condition or phenotype.

As used herein, the term “cancer” refers to any of various types of malignant neoplasms, most of which invade surrounding tissues, may metastasize to several sites and are likely to recur after attempted removal and to cause death of the patient unless adequately treated. As used herein, neoplasia comprises cancer. Representative cancers include, for example, squamous-cell carcinoma, basal cell carcinoma, adenocarcinoma, hepatocellular carcinomas, and renal cell carcinomas, cancer of the bladder, bowel, breast, cervix, colon, esophagus, head, kidney, liver, lung, neck, ovary, pancreas, prostate, and stomach; leukemias, including non-acute and acute leukemias, such as acute myelogenous leukemia, acute lymphocytic leukemia, acute promyelocytic leukemia (APL), acute T-cell lymphoblastic leukemia, T-lineage acute lymphoblastic leukemia (T-ALL), adult T-cell leukemia, basophilic leukemia, eosinophilic leukemia, granulocytic leukemia, hairy cell leukemia, leukopenic leukemia, lymphatic leukemia, lymphoblastic leukemia, lymphocytic leukemia, megakaryocytic leukemia, micromyeloblastic leukemia, monocytic leukemia, neutrophilic leukemia and stem cell leukemia; benign and malignant lymphomas, particularly Burkitt's lymphoma and Non-Hodgkin's lymphoma; benign and malignant melanomas; myeloproliferative diseases; sarcomas, including Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, glioblastomas, neuroblastomas, ganglioneuromas, gangliogliomas, medulloblastomas, pineal cell tumors, meningiomas, meningeal sarcomas, neurofibromas, and Schwannomas; bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma; carcinosarcoma, Hodgkin's disease, Wilms'tumor and teratocarcinomas, among others, which may be treated by at least one compound of the present invention.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The present invention is based in part on the discovery that inhibition of SARM1 signaling pathways prevents cilia disassembly. Accordingly, in some embodiments the invention is directed towards compositions and methods for preventing cilia disassembly. The present invention is further based on the discovery that prevention of cilia disassembly by SARM1 signaling pathways is effective in treating a variety of diseases and disorders associated with cilia disassembly or dysfunction (i.e., ciliopathies). Accordingly, in some embodiments, the invention is directed to treating a disease or disorder associated with cilia disassembly or dysfunction (e.g., focal malformation of cortical development (FMCD) disorders).

In some embodiments, the invention is directed to the use of a composition comprising at least one selected from the group consisting of an inhibitor of coagulation factor II receptor (F2R), sterile alpha and TIR motif containing 1 (SARM1), ryanodine receptor 1 (RyR1), ryanodine receptor 2 (RyR2), ryanodine receptor 3 (RyR3), Ras homolog member A (RhoA), Rho-associated protein kinase 1 (ROCK1), and Rho-associated protein kinase 2 (ROCK2).

### Compositions

In some embodiments, the present invention provides compositions comprising at least one inhibitor of cilia disassembly. In some embodiments, the composition comprises at least one inhibitor of at least one protein in the SARM1-cilia disassembly pathway. In some embodiments, the at least one protein in the SARM1-cilia disassembly pathway is at least one protein selected from the group consisting of F2R, SARM1, RyR1, RyR2, RyR3, RhoA, ROCK1, and ROCK2. An inhibitor of a protein in the SARM1-cilia disassembly pathway is any compound, molecule, or agent that reduces, inhibits, or prevents the function of a protein in the SARM1-cilia disassembly pathway. For example, an inhibitor of a protein in the SARM1-cilia disassembly pathway is any compound, molecule, or agent that reduces expression, activity, or both of a protein in the SARM1-cilia disassembly pathway. Exemplary inhibitors include, but are not limited, to small molecules, isolated nucleic acids, vectors, isolated peptides, peptide mimetics, and the like.

In some embodiments, the composition comprises at least one inhibitor of at least one protein upstream of SARM1 in the SARM1-cilia disassembly pathway. In some embodiments, the composition comprises at least one inhibitor of at least one protein downstream of SARM1 in the SARM1-cilia disassembly pathway. In some embodiments, the composition comprises at least one inhibitor of SARM1. In some embodiments, the composition comprises at least one selected from the group consisting of at least one inhibitor of at least one protein upstream of SARM1 in the SARM1-cilia disassembly pathway; at least one inhibitor of at least one protein downstream of SARM1 in the SARM1-cilia disassembly pathway; and at least one inhibitor of SARM1.

### Small Molecule Inhibitors

In various embodiments, the inhibitor is a small molecule. When the inhibitor is a small molecule, a small molecule may be obtained using standard methods known to the skilled artisan. Such methods include chemical organic synthesis or biological means. Biological means include purification from a biological source, recombinant synthesis and in vitro translation systems, using methods well known in the art. In one embodiment, a small molecule inhibitor of the invention comprises an organic molecule, inorganic molecule, biomolecule, synthetic molecule, and the like.

Combinatorial libraries of molecularly diverse chemical compounds potentially useful in treating a variety of diseases and conditions are well known in the art as are method of making the libraries. The method may use a variety of techniques well-known to the skilled artisan including solid phase synthesis, solution methods, parallel synthesis of single compounds, synthesis of chemical mixtures, rigid core structures, flexible linear sequences, deconvolution strategies, tagging techniques, and generating unbiased molecular landscapes for lead discovery vs. biased structures for lead development.

In a general method for small library synthesis, an activated core molecule is condensed with a number of building blocks, resulting in a combinatorial library of covalently linked, core-building block ensembles. The shape and rigidity of the core determines the orientation of the building blocks in shape space. The libraries can be biased by changing the core, linkage, or building blocks to target a characterized biological structure (“focused libraries”) or synthesized with less structural bias using flexible cores.

The small molecule and small molecule compounds described herein may be present as salts even if salts are not depicted, and it is understood that the invention embraces all salts and solvates of the inhibitors depicted here, as well as the non-salt and non-solvate form of the inhibitors, as is well understood by the skilled artisan. In some embodiments, the salts of the inhibitors of the invention are pharmaceutically acceptable salts.

Where tautomeric forms may be present for any of the inhibitors described herein, each and every tautomeric form is intended to be included in the present invention, even though only one or some of the tautomeric forms may be explicitly depicted. For example, when a 2-hydroxypyridyl moiety is depicted, the corresponding 2-pyridone tautomer is also intended.

The invention also includes any or all of the stereochemical forms, including any enantiomeric or diastereomeric forms of the inhibitors described. The recitation of the structure or name herein is intended to embrace all possible stereoisomers of inhibitors depicted. All forms of the inhibitors are also embraced by the invention, such as crystalline or non-crystalline forms of the inhibitors. Compositions comprising an inhibitor of the invention are also intended, such as a composition of substantially pure inhibitor, including a specific stereochemical form thereof, or a composition comprising mixtures of inhibitors of the invention in any ratio, including two or more stereochemical forms, such as in a racemic or non-racemic mixture.

In one embodiment, the small molecule inhibitor of the invention comprises an analog or derivative of an inhibitor described herein.

In one embodiment, the small molecules described herein are candidates for derivatization. As such, in certain instances, the analogs of the small molecules described herein that have modulated potency, selectivity, and solubility are included herein and provide useful leads for drug discovery and drug development. Thus, in certain instances, during optimization new analogs are designed considering issues of drug delivery, metabolism, novelty, and safety.

In some instances, small molecule inhibitors described herein are derivatized/analoged as is well known in the art of combinatorial and medicinal chemistry. The analogs or derivatives can be prepared by adding and/or substituting functional groups at various locations. As such, the small molecules described herein can be converted into derivatives/analogs using well known chemical synthesis procedures. For example, all of the hydrogen atoms or substituents can be selectively modified to generate new analogs. Also, the linking atoms or groups can be modified into longer or shorter linkers with carbon backbones or hetero atoms. Also, the ring groups can be changed so as to have a different number of atoms in the ring and/or to include hetero atoms. Moreover, aromatics can be converted to cyclic rings, and vice versa. For example, the rings may be from 5-7 atoms, and may be homocycles or heterocycles.

As used herein, the term “analog,” “analogue,” or “derivative” is meant to refer to a chemical compound or molecule made from a parent compound or molecule by at least one chemical reaction. As such, an analog can be a structure having a structure similar to that of the small molecule inhibitors described herein or can be based on a scaffold of a small molecule inhibitor described herein, but differing from it in respect to certain components or structural makeup, which may have a similar or opposite action metabolically. An analog or derivative of any of a small molecule inhibitor in accordance with the present invention can be used to reduce skin pigmentation.

In one embodiment, the small molecule inhibitors described herein can independently be derivatized/analoged by modifying hydrogen groups independently from each other into other substituents. That is, each atom on each molecule can be independently modified with respect to the other atoms on the same molecule. Any traditional modification for producing a derivative/analog can be used. For example, the atoms and substituents can be independently comprised of hydrogen, an alkyl, aliphatic, straight chain aliphatic, aliphatic having a chain hetero atom, branched aliphatic, substituted aliphatic, cyclic aliphatic, heterocyclic aliphatic having at least one hetero atom, aromatic, heteroaromatic, polyaromatic, polyamino acid, peptide, polypeptide, combination thereof, halogen, halo-substituted aliphatic, and the like. Additionally, any ring group on a compound can be derivatized to increase and/or decrease ring size as well as change the backbone atoms to carbon atoms or hetero atoms.

In some embodiments, the composition comprises at least one inhibitor of at least one protein upstream of SARM1 in the SARM1-cilia disassembly pathway. In various embodiments, the composition comprises one or inhibitors of at least one selected from the group consisting of lysophosphatidic acid receptor 1 (LPAR1), mammalian target of rapamycin complex 1 (mTORC1), thrombin, and coagulation factor II receptor (F2R). In some embodiments, the at least one inhibitor is at least one inhibitor of LPAR1. In some embodiments, the at least one inhibitor of LPAR1 is at least one LPAR1 antagonist. In some embodiments, the at least one LPAR1 antagonist is selected from the group consisting of AM966, BMS-986020, BMS-986278, Ki16425, analogs, derivatives, and prodrugs thereof, and pharmaceutically acceptable salts and hydrates thereof. In some embodiments, the composition comprises at least one inhibitor of F2R. In some embodiments, the at least one inhibitor of F2R is at least one F2R antagonist. In some embodiments, the at least one F2R antagonist is at least one selected from the group consisting of vorapaxar, atopaxar, SCH-79797, analogs, derivatives, and prodrugs thereof, and pharmaceutically acceptable salts and hydrates thereof. In some embodiments, the composition comprises at least one thrombin inhibitor. In some embodiments, the at least one thrombin inhibitor is at least one selected from the group consisting of argatroban, bivalirudin, dabigatran, lepirudin, desirudin, analogs, derivatives, and prodrugs thereof, and pharmaceutically acceptable salts and hydrates thereof. In some embodiments, the composition comprises at least one mTORC1 inhibitor. In some embodiments, the at least one mTORC1 inhibitor is at least one selected from the group consisting of dactolisib, rapamycin, everolimus, AZD8055, temsirolimus, PI-103, KU-0063794, torkinib, ridaforolimus, sapanisertib, voxtalisib, torin 1, omipalisib, OSI-027, PF-0461502, apitolisib, GSK1059615, gedatolisib, WYE-354, vistusertib, torin 2, WYE-125132, PP121, WYE-687, WAY-600, ETP-46464, GDC-0349, XL388, SF2523, CZ415, onatasertib, voxtalisib, zotarolimus, tacrolimus, BGT226, palomid 529, samotolisib, and chrysophanic acid.

In some embodiments, a composition of the present invention comprises at least one small molecule inhibitor of at least one protein downstream of SARM1 in the SARM1-cilia disassembly pathway. In various embodiments, the composition comprises at least one inhibitor of at least one selected from the group consisting of ryanodine receptor 1 (RyR1), ryanodine receptor 2 (RyR2), ryanodine receptor 3 (RyR3), Ras homolog member A (RhoA), Rho-associated protein kinase 1 (ROCK1), and Rho-associated protein kinase 2 (ROCK2). In some embodiments, the at least one inhibitor is at least one inhibitor of RyR1. In some embodiments, the at least one inhibitor of RyR1 is at least one selected from the group consisting of dantrolene, 6,7-(methylenedioxy)-1-octyl-4-quinolone-3-carboxylic acid, analogs, derivatives, and prodrugs thereof, and pharmaceutically acceptable salts and hydrates thereof. In some embodiments, the at least one inhibitor of RyR2 is at least one selected from the group consisting of dantrolene, analogs, derivatives, and prodrugs thereof, and pharmaceutically acceptable salts and hydrates thereof. In some embodiments, the at least one inhibitor of RyR3 is at least one selected from the group consisting of dantrolene, analogs, derivatives, and prodrugs thereof, and pharmaceutically acceptable salts and hydrates thereof. In some embodiments, the at least one inhibitor of RhoA is at least one selected from the group consisting of NSC 23766, zoledronic acid, EHT 1864, CCF-1423, ML141, analogs, derivatives, and prodrugs thereof, and pharmaceutically acceptable salts and hydrates thereof. In some embodiments, the at least one inhibitor of ROCK1 is at least one selected from the group consisting of thiazovivin, Y-27632,DJ4, GSK269962A, WAY-624704, RKI-1447, Chroman 1, azaindole 1, ATI13148, GSK429286A, Y-39983, ripasudil, hydroxyfasudil, netarsudil, ZINC00881524, analogs, derivatives, and prodrugs thereof, and pharmaceutically acceptable salts and hydrates thereof. In some embodiments, the at least one inhibitor of ROCK2 is at least one selected from the group consisting of thiazovivin, Y-27632, DJ4, GSK269962A, WAY-624704, RKI-1447, Chroman 1, azaindole 1, ATI13148, GSK429286A, Y-39983, ripasudil, fasudil, hydroxyfasudil, netarsudil, ZINC00881524, H-1152, belumosudil, analogs, derivatives, and prodrugs thereof, and pharmaceutically acceptable salts and hydrates thereof.

In some embodiments, a composition of the present invention comprises at least one small molecule inhibitor of SARM1. In some embodiments, the at least one inhibitor of SARM1 is at least one selected from the group consisting of DSRM-3716, dehydronitrosonisodipine (dHNN), MY-9B, WX-02-37, EV-99, WX-02-34, WX-02-35, 3-oxo-5-((2-(trifulromethyl)phenyl)amino)-2,3-dihydroisothiazole-4-carbonitrile, 5-((2-chlorophenyl)amino)-3-oxo-2,3-dihydroisothiazole-4-carbonitrile, 5-((2-methylsulfonyl)phenyl)amino)-3-oxo-2,3-dihydroisothiazole-4-carbonitrile, 5-((6-chloroquinolin-5-yl)amino)-2-methyl-3-oxo-2,3-dihydroisothiazole-4-carbonitrile, 5-((6-methoxy-2-methylquinolin-5-yl)amino)-2-methyl-3-oxo-2,3-dihydroisothiazole-4-carbonitrile, nicotinic acid mononucleotide (NMN), 2-(4-Pyridinyl)-N-(2,2,2-trifluoroethyl)-1-pyrrolidinecarboxamide, 1-(Pyridin-4-ylmethyl)-3-(2,2,2-trifluoroethyl)urea, (S)-1-Methyl-1-(1-(pyridin-4-yl)ethyl)-3-(2,2,2-trifluoroethyl)urea, (R)-1-methyl-1-(1-(pyridin-4-yl)ethyl)-3-(2,2,2-trifluoroethyl)urea, (S)-1-methyl-1-(1-phenylethyl)-3-(2,2,2-trifluoroethyl)urea, 3-(3-chlorophenyl)-N-(4-methyl-3-(pyridin-4-yl)-1H-pyrazol-5-yl)propenamide, 3-(4-Chlorophenyl)-N-(4-methyl-3-(pyridin-4-yl)-1H-pyrazol-5-yl)propenamide, analogs, derivatives, and prodrugs thereof, and pharmaceutically acceptable salts and hydrates thereof.

In some embodiments, the composition comprises at least one small molecule inhibitor of selected from the group consisting of at least one protein upstream of SARM1 in the SARM1-cilia disassembly pathway, at least one small molecule inhibitor of at least one protein downstream of SARM1 in the SARM1-cilia disassembly pathway, and at least one small molecule inhibitor of SARM1. In some embodiments, the composition comprises at least one small molecule inhibitor of at least one protein upstream of SARM1 in the SARM1-cilia disassembly pathway and at least one small molecule inhibitor of at least one protein downstream of SARM1 in the SARM1-cilia disassembly pathway. In some embodiments, the composition comprises at least one small molecule inhibitor of at least one protein upstream of SARM1 in the SARM1-cilia disassembly pathway and at least one small molecule inhibitor of SARM1. In some embodiments, the composition comprises at least one small molecule inhibitor of SARM1 and at least one small molecule inhibitor of at least one protein downstream of SARM1 in the SARM1-cilia disassembly pathway. In some embodiments, the composition comprises at least one small molecule inhibitor of at least one protein upstream of SARM1 in the SARM1-cilia disassembly pathway, at least one small molecule inhibitor of at least one protein downstream of SARM1 in the SARM1-cilia disassembly pathway, and at least one small molecule inhibitor of SARM1. In some embodiments, a composition of the present invention comprises at least one small molecule inhibitor selected from the group consisting of at least one small molecule inhibitor of at least one protein upstream of SARM1 in the SARM1-cilia disassembly pathway selected from the group consisting of AM966, BMS-986020, BMS-986278, vorapaxar, atopaxar, SCH-79797, argatroban, bivalirudin, dabigatran, lepirudin, desirudin, dactolisib, rapamycin, everolimus, AZD8055, temsirolimus, PI-103, KU-0063794, torkinib, ridaforolimus, sapanisertib, voxtalisib, torin 1, omipalisib, OSI-027, PF-0461502, apitolisib, GSK1059615, gedatolisib, WYE-354, vistusertib, torin 2, WYE-125132, PP121, WYE-687, WAY-600, ETP-46464, GDC-0349, XL388, SF2523, CZ415, onatasertib, voxtalisib, zotarolimus, tacrolimus, BGT226, palomid 529, samotolisib, chrysophanic acid, dactolisib, rapamycin, everolimus, AZD8055, temsirolimus, PI-103, KU-0063794, torkinib, ridaforolimus, sapanisertib, voxtalisib, torin 1, omipalisib, OSI-027, PF-0461502, apitolisib, GSK1059615, gedatolisib, WYE-354, vistusertib, torin 2, WYE-125132, PP121, WYE-687, WAY-600, ETP-46464, GDC-0349, XL388, SF2523, CZ415, onatasertib, voxtalisib, zotarolimus, tacrolimus, BGT226, palomid 529, samotolisib, chrysophanic acid, analogs, derivatives, and prodrugs thereof, and pharmaceutically acceptable salts and hydrates thereof; at least one small molecule inhibitor of at least one protein downstream of SARM1 in the SARM1-cilia disassembly pathway selected from the group consisting of dantrolene, 6,7-(methylenedioxy)-1-octyl-4-quinolone-3-carboxylic acid, NSC 23766, zoledronic acid, EHT 1864, CCF-1423, ML141, thiazovivin, Y-27632,DJ4, GSK269962A, WAY-624704, RKI-1447, Chroman 1, azaindole 1, ATI13148, GSK429286A, Y-39983, ripasudil, hydroxyfasudil, netarsudil, ZINC00881524, H-1152, belumosudil, analogs, derivatives, and prodrugs thereof, and pharmaceutically acceptable salts and hydrates thereof; and at least one small molecule inhibitor of SARM1 selected from the group consisting of DSRM-3716, dehydronitrosonisodipine (dHNN), MY-9B, WX-02-37, EV-99, WX-02-34, WX-02-35, 3-oxo-5-((2-(trifulromethyl)phenyl)amino)-2,3-dihydroisothiazole-4-carbonitrile, 5-((2-chlorophenyl)amino)-3-oxo-2,3-dihydroisothiazole-4-carbonitrile, 5-((2- methylsulfonyl)phenyl)amino)-3-oxo-2,3-dihydroisothiazole-4-carbonitrile, 5-((6-chloroquinolin-5-yl)amino)-2-methyl-3-oxo-2,3-dihydroisothiazole-4-carbonitrile, 5-((6-methoxy-2-methylquinolin-5-yl)amino)-2-methyl-3-oxo-2,3-dihydroisothiazole-4-carbonitrile, nicotinic acid mononucleotide (NMN), 2-(4-Pyridinyl)-N-(2,2,2-trifluoroethyl)-1-pyrrolidinecarboxamide, 1-(Pyridin-4-ylmethyl)-3-(2,2,2-trifluoroethyl)urea, (S)-1-Methyl-1-(1-(pyridin-4- yl)ethyl)-3-(2,2,2-trifluoroethyl)urea, (R)-1-methyl-1-(1-(pyridin-4-yl)ethyl)-3-(2,2,2-trifluoroethyl)urea, (S)-1-methyl-1-(1-phenylethyl)-3-(2,2,2-trifluoroethyl)urea, 3-(3-chlorophenyl)-N-(4-methyl-3-(pyridin-4-yl)-1H-pyrazol-5-yl)propenamide, 3-(4-Chlorophenyl)-N-(4-methyl-3-(pyridin-4-yl)-1H-pyrazol-5-yl)propenamide, analogs, derivatives, and prodrugs thereof, and pharmaceutically acceptable salts and hydrates thereof.

In some embodiments, a composition of the present invention further comprises at least one calcium (Ca²⁺) chelator. In some embodiments, the at least one calcium chelator is at least one selected form the group consisting of 1,2-bis(2-aminophenoxy)ethane-N,N,N′,N′-tetraacetic acid tetrakis(acetoxymethyl ester) (BAPTA-AM), ethylenediaminetetraacetic acid (EDTA), ethyleneglycoltetraacetic acid (EGTA), diethylenetriaminepentaacetate (DTPA), hydroxyethylethylenediaminetriacetic acid (HEEDTA), diaminocyclohexanetetraacetic acid (CDRA), 1,2-bis(2-aminophenoxy)ethane-N,N,N′,N′-tetraacetic acid (BAPTA), analogs, derivatives, and prodrugs thereof, and pharmaceutically acceptable salts and hydrates thereof.

The compounds of the invention may possess at least one stereocenter, and each stereocenter may exist independently in either the R or S configuration. In one embodiment, compounds described herein are present in optically active or racemic forms. It is to be understood that the compounds described herein encompass racemic, optically active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically active starting materials, chiral synthesis, or chromatographic separation using a chiral stationary phase. In one embodiment, a mixture of at least one isomer is utilized as the therapeutic compound described herein. In another embodiment, compounds described herein contain at least one chiral center. These compounds are prepared by any means, including stereoselective synthesis, enantioselective synthesis and/or separation of a mixture of enantiomers /d/ or diastereomers. Resolution of compounds and isomers thereof is achieved by any means including, by way of non-limiting example, chemical processes, enzymatic processes, fractional crystallization, distillation, and chromatography.

In one embodiment, compounds described herein are prepared as prodrugs. A “prodrug” refers to an agent that is converted into the parent drug in vivo. In one embodiment, upon in vivo administration, a prodrug is chemically converted to the biologically, pharmaceutically or therapeutically active form of the compound. In another embodiment, a prodrug is enzymatically metabolized by at least one step or process to the biologically, pharmaceutically or therapeutically active form of the compound.

### Nucleic Acid Inhibitors

In other embodiments, the invention includes an isolated nucleic acid. In some embodiments, the inhibitor is an siRNA, shRNA or antisense molecule, which inhibits a protein in the SARM1-cilia disassembly pathway. In one embodiment, the nucleic acid comprises a promoter/regulatory sequence such that the nucleic acid is capable of directing expression of the nucleic acid. Thus, the invention encompasses expression vectors and methods for the introduction of exogenous DNA into cells with concomitant expression of the exogenous DNA in the cells such as those described, for example, in Sambrook et al. (2012, Molecular Cloning: A Laboratory Manual, Cold Spring Harbor Laboratory, New York), and in Ausubel et al. (1997, Current Protocols in Molecular Biology, John Wiley & Sons, New York) and as described elsewhere herein.

In another aspect of the invention, a protein in the SARM1-cilia disassembly pathway can be inhibited by way of inactivating and/or sequestering a protein in the SARM1-cilia disassembly pathway. As such, inhibiting the activity of a protein in the SARM1-cilia disassembly pathway can be accomplished by using a transdominant negative mutant.

In one embodiment, siRNA or shRNA is used to decrease the level of a protein in the SARM1-cilia disassembly pathway. RNA interference (RNAi) is a phenomenon in which the introduction of double-stranded RNA (dsRNA) into a diverse range of organisms and cell types causes disassembly of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific disassembly of mRNA results in gene silencing. See, for example, U.S. Pat. No. 6,506,559; Fire et al., 1998, Nature 391(19): 306-311; Timmons et al., 1998, Nature 395: 854; Montgomery et al., 1998, TIG 14 (7): 255-258; David R. Engelke, Ed., RNA Interference (RNAi) Nuts & Bolts of RNAi Technology, DNA Press, Eagleville, PA (2003); and Gregory J. Hannon, Ed., RNAi A Guide to Gene Silencing, Cold Spring Harbor Laboratory Press, Cold Spring Harbor, NY (2003). Soutschek et al. (2004, Nature 432: 173-178) describe a chemical modification to siRNAs that aids in intravenous systemic delivery. Optimizing siRNAs involves consideration of overall G/C content, C/T content at the termini, Tm and the nucleotide content of the 3′ overhang. See, for instance, Schwartz et al., 2003, Cell, 115: 199-208 and Khvorova et al., 2003, Cell 115: 209-216. Therefore, the present invention also includes methods of decreasing levels of a protein in the SARM1-cilia disassembly pathway using RNAi technology.

In another aspect, the invention includes a vector comprising an siRNA or antisense polynucleotide. In one embodiment, the siRNA or antisense polynucleotide is capable of inhibiting the expression of a target polypeptide, wherein the target polypeptide is selected from the group consisting of p21 and telomerase. The incorporation of a desired polynucleotide into a vector and the choice of vectors is well-known in the art as described in, for example, Sambrook et al. (2012), and in Ausubel et al. (1997), and elsewhere herein.

In certain embodiments, the expression vectors described herein encode a short hairpin RNA (shRNA) inhibitor. shRNA inhibitors are well known in the art and are directed against the mRNA of a target, thereby decreasing the expression of the target. In certain embodiments, the encoded shRNA is expressed by a cell, and is then processed into siRNA. For example, in certain instances, the cell possesses native enzymes (e.g., dicer) that cleaves the shRNA to form siRNA. The siRNA, shRNA, or antisense polynucleotide can be cloned into a number of types of vectors as described elsewhere herein. For expression of the siRNA or antisense polynucleotide, at least one module in each promoter functions to position the start site for RNA synthesis.

In order to assess the expression of the siRNA, shRNA, or antisense polynucleotide, the expression vector to be introduced into a cell can also contain either a selectable marker gene or a reporter gene or both to facilitate identification and selection of expressing cells from the population of cells sought to be transfected or infected using a viral vector. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neomycin resistance and the like.

Therefore, in another aspect, the invention relates to a vector, comprising the nucleotide sequence of the invention or the construct of the invention. The choice of the vector will depend on the host cell in which it is to be subsequently introduced. In a particular embodiment, the vector of the invention is an expression vector. Suitable host cells include a wide variety of prokaryotic and eukaryotic host cells. In specific embodiments, the expression vector is selected from the group consisting of a viral vector, a bacterial vector and a mammalian cell vector.

Prokaryote- and/or eukaryote-vector based systems can be employed for use with the present invention to produce polynucleotides, or their cognate polypeptides. Many such systems are commercially and widely available.

Further, the expression vector may be provided to a cell in the form of a viral vector. Viral vector technology is well known in the art and is described, for example, in Sambrook et al. (2012), and in Ausubel et al. (1997), and in other virology and molecular biology manuals.

Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and at least one selectable marker. (See, e.g., WO 01/96584; WO 01/29058; and U.S. Pat. No. 6,326,193.

By way of illustration, the vector in which the nucleic acid sequence is introduced can be a plasmid which is or is not integrated in the genome of a host cell when it is introduced in the cell. Illustrative, non-limiting examples of vectors in which the nucleotide sequence of the invention or the gene construct of the invention can be inserted include a tet-on inducible vector for expression in eukaryote cells.

The vector may be obtained by conventional methods known by persons skilled in the art (Sambrook et al., 2012). In a particular embodiment, the vector is a vector useful for transforming animal cells.

In one embodiment, the recombinant expression vectors may also contain nucleic acid molecules which encode a peptide or peptidomimetic inhibitor of invention, described elsewhere herein.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (U.S. Pat. Nos. 4,683,202, 5,928,906). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression, for example, see Sambrook et al. (2012). The promoters employed may be constitutive, tissue-specific, inducible, and/or useful under the appropriate conditions to direct high-level expression of the introduced DNA segment, such as is advantageous in the large-scale production of recombinant proteins and/or peptides.

The promoter may be heterologous or endogenous.

The recombinant expression vectors may also contain a selectable marker gene which facilitates the selection of transformed or transfected host cells. Suitable selectable marker genes are genes encoding proteins such as G418 and hygromycin which confer resistance to certain drugs, β-galactosidase, chloramphenicol acetyltransferase, firefly luciferase, or an immunoglobulin or portion thereof such as the Fc portion of an immunoglobulin, for example, IgG. The selectable markers may be introduced on a separate vector from the nucleic acid of interest.

Following the generation of the siRNA polynucleotide, a skilled artisan will understand that the siRNA polynucleotide will have certain characteristics that can be modified to improve the siRNA as a therapeutic compound. Therefore, the siRNA polynucleotide may be further designed to resist disassembly by modifying it to include phosphorothioate, or other linkages, methylphosphonate, sulfone, sulfate, ketyl, phosphorodithioate, phosphoramidate, phosphate esters, and the like (see, e.g., Agrwal et al., 1987, Tetrahedron Lett. 28: 3539-3542; Stec et al., 1985 Tetrahedron Lett. 26: 2191-2194; Moody et al., 1989 Nucleic Acids Res. 12: 4769-4782; Eckstein, 1989 Trends Biol. Sci. 14: 97-100; Stein, In: Oligodeoxynucleotides. Antisense Inhibitors of Gene Expression, Cohen, ed., Macmillan Press, London, pp. 97-117 (1989)).

Any polynucleotide may be further modified to increase its stability in vivo. Possible modifications include, but are not limited to, the addition of flanking sequences at the 5′ and/or 3′ ends; the use of phosphorothioate or 2′ O-methyl rather than phosphodiester linkages in the backbone; and/or the inclusion of nontraditional bases such as inosine, queosine, and wybutosine and the like, as well as acetyl-methyl-, thio-and other modified forms of adenine, cytidine, guanine, thymine, and uridine.

In one embodiment of the invention, an antisense nucleic acid sequence which is expressed by a plasmid vector is used to inhibit expression of a protein in the SARM1-cilia disassembly pathway. The antisense expressing vector is used to transfect a mammalian cell or the mammal itself, thereby causing reduced endogenous expression of a protein in the SARM1-cilia disassembly pathway.

Antisense molecules and their use for inhibiting gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262: 40). In the cell, antisense nucleic acids hybridize to the corresponding mRNA, forming a double-stranded molecule thereby inhibiting the translation of genes.

The use of antisense methods to inhibit the translation of genes is known in the art, and is described, for example, in Marcus-Sakura (1988, Anal. Biochem. 172: 289). Such antisense molecules may be provided to the cell via genetic expression using DNA encoding the antisense molecule as taught by Inoue, 1993, U.S. Pat. No. 5,190,931.

Alternatively, antisense molecules of the invention may be made synthetically and then provided to the cell. In one embodiment, antisense oligomers may have between about 10 to about 30 nucleotides. In one embodiment, antisense oligomers may have about 15 nucleotides. In one embodiment, antisense oligomers having 10-30 nucleotides are easily synthesized and introduced into a target cell. Synthetic antisense molecules contemplated by the invention include oligonucleotide derivatives known in the art which have improved biological activity compared to unmodified oligonucleotides (see U.S. Pat. No. 5,023,243).

In one embodiment of the invention, a ribozyme is used to inhibit expression of a protein in the SARM1-cilia disassembly pathway. Ribozymes useful for inhibiting the expression of a target molecule may be designed by incorporating target sequences into the basic ribozyme structure which are complementary, for example, to the mRNA sequence encoding a protein in the SARM1-cilia disassembly pathway. Ribozymes targeting a protein in the SARM1-cilia disassembly pathway may be synthesized using commercially available reagents (Applied Biosystems, Inc., Foster City, CA) or they may be genetically expressed from DNA encoding them.

In one embodiment, the inhibitor of a protein in the SARM1-cilia disassembly pathway may comprise at least one component of a CRISPR-Cas system, where a guide RNA (gRNA) targeted to a gene encoding a protein in the SARM1-cilia disassembly pathway, and a CRISPR-associated (Cas) peptide form a complex to induce mutations within the targeted gene. In one embodiment, the inhibitor comprises a gRNA or a nucleic acid molecule encoding a gRNA. In one embodiment, the inhibitor comprises a Cas peptide or a nucleic acid molecule encoding a Cas peptide.

### Polypeptide Inhibitors

In other related aspects, the invention includes an isolated peptide inhibitor that inhibits a protein in the SARM1-cilia disassembly pathway. For example, in one embodiment, the peptide inhibitor of the invention inhibits a protein in the SARM1-cilia disassembly pathway directly by binding to a protein in the SARM1-cilia disassembly pathway thereby preventing the normal functional activity of a protein in the SARM1-cilia disassembly pathway. In another embodiment, the peptide inhibitor of the invention inhibits a protein in the SARM1-cilia disassembly pathway by competing with endogenous a protein in the SARM1-cilia disassembly pathway. In yet another embodiment, the peptide inhibitor of the invention inhibits the activity of a protein in the SARM1-cilia disassembly pathway by acting as a transdominant negative mutant.

The variants of the polypeptides according to the present invention may be (i) one in which at least one of the amino acid residues are substituted with a conserved or non-conserved amino acid residue and such substituted amino acid residue may or may not be one encoded by the genetic code, (ii) one in which there is at least one modified amino acid residue, e.g., residues that are modified by the attachment of substituent groups, (iii) one in which the polypeptide is an alternative splice variant of the polypeptide of the present invention, (iv) fragments of the polypeptides and/or (v) one in which the polypeptide is fused with another polypeptide, such as a leader or secretory sequence or a sequence which is employed for purification (for example, His-tag) or for detection (for example, Sv5 epitope tag). The fragments include polypeptides generated via proteolytic cleavage (including multi-site proteolysis) of an original sequence.

Variants may be post-translationally, or chemically modified. Such variants are deemed to be within the scope of those skilled in the art from the teaching herein.

### Antibody Inhibitors

The invention also contemplates an inhibitor of a protein in the SARM1-cilia disassembly pathway comprising an antibody, or antibody fragment, specific for a protein in the SARM1-cilia disassembly pathway. That is, the antibody can inhibit a protein in the SARM1-cilia disassembly pathway to provide a beneficial effect.

The antibodies may be intact monoclonal or polyclonal antibodies, and immunologically active fragments (e.g., a Fab or (Fab)₂ fragment), an antibody heavy chain, an antibody light chain, humanized antibodies, a genetically engineered single chain Fv molecule (Ladner et al, U.S. Pat. No. 4,946,778), or a chimeric antibody, for example, an antibody which contains the binding specificity of a murine antibody, but in which the remaining portions are of human origin. Antibodies including monoclonal and polyclonal antibodies, fragments and chimeras, may be prepared using methods known to those skilled in the art.

Antibodies can be prepared using intact polypeptides or fragments containing an immunizing antigen of interest. The polypeptide or oligopeptide used to immunize an animal may be obtained from the translation of RNA or synthesized chemically and can be conjugated to a carrier protein, if desired. Suitable carriers that may be chemically coupled to peptides include bovine serum albumin and thyroglobulin, keyhole limpet hemocyanin. The coupled polypeptide may then be used to immunize the animal (e.g., a mouse, a rat, or a rabbit).

### Methods

In some embodiments, the disclosure provides methods of preventing cilia disassembly. In one embodiment, the method comprises administering to the subject an effective amount of a composition comprising at least one inhibitor of cilia disassembly. In some embodiments, the method comprises administering a composition comprising at least one inhibitor of at least one protein in the SARM1-cilia disassembly pathway.

In some embodiments, the methods are useful in treating a disease or disorder associated with cilia disassembly or dysfunction (i.e., a ciliopathy) in a subject. In some embodiments, the disease or disorder associated with cilia disassembly or dysfunction is selected from the group consisting of focal malformations of cortical development (FMCD), focal cortical dysplasia (FCD), Joubert syndrome, orofaciodigital syndrome, Leber's congenital amaurosis, Bardet-Biedl syndrome, Alström syndrome, Jeune asphyxiating thoracic dystrophy, Ellis van Creveld syndrome, Sensenbrenner syndrome, polycystic kidney disease, cystic renal dysplasia, nephronophthisis, McKusick-Kaufman syndrome, Meckel-Gruber syndrome, primary ciliary dyskinesia, Senior-Løken syndrome, Short rib-polydactyly syndrome, acrocallosal syndrome, acrometlic frontonasal dysostosis, Arima syndrome, Biemond syndrome, COACH syndrome, conorenal syndrome, Greig cephalopolysyndactyly syndrome, hydrolethalus syndrome, Johanson-Blizzard syndrome, Neu-Laxova syndrome, Optiz G/BBB syndrome, Pallister-Hall syndrome, papillorenal syndrome, renal-hepatic-pancreatic dysplasia, periventricular heterotopia, and mitral valve prolapse.

In some embodiments, the methods are useful in treating cancer. In some embodiments, the cancer is a cancer associated with aberrant loss of cilia. Examples of cancers associated with aberrant loss of cilia include, but are not limited to, glioblastoma, breast cancer, and melanoma. In some embodiments, the cancer is a cancer which requires cycles of cilia assembly and/or disassembly for growth. Examples of cancers requiring cycles of cilia assembly and/or disassembly include, but are not limited to, medulloblastoma.

In some embodiments, the disclosure provides methods comprising administering to the subject an effective amount of a composition according to the present invention.

The compositions of the disclosure may be administered to a patient or subject in need in a wide variety of ways. Modes of administration include intraoperatively intravenous, intravascular, intramuscular, subcutaneous, intracerebral, intraperitoneal, soft tissue injection, surgical placement, arthroscopic placement, and percutaneous insertion, e.g., direct injection, cannulation, or catheterization. Any administration may be a single application of a composition of invention or multiple applications. Administrations may be to single site or to more than one site in the individual to be treated. Multiple administrations may occur essentially at the same time or separated in time.

In some embodiments, the compositions may be administered in combination with at least one additional composition. In certain embodiments, the composition of the present invention may be combined with at least one additional composition of the present invention.

### Pharmaceutical Compositions and Formulations

The invention also encompasses the use of pharmaceutical compositions to practice the methods of the invention. Such a pharmaceutical composition may consist of at least one composition of the invention or a salt thereof in a form suitable for administration to a subject, or the pharmaceutical composition may comprise at least one composition of the invention or a salt thereof, and at least one pharmaceutically acceptable carrier, at least one additional ingredient, or some combination of these. The compound or conjugate may be present in the pharmaceutical composition in the form of a physiologically acceptable salt, such as in combination with a physiologically acceptable cation or anion, as is well known in the art.

Pharmaceutical compositions that are useful in the methods of the invention may be suitably developed for oral, rectal, vaginal, parenteral, topical, pulmonary, intranasal, buccal, sublingual, ophthalmic, or another route of administration. A composition useful within the methods of the invention may be directly administered to the skin, vagina or any other tissue of a mammal. Other contemplated formulations include liposomal preparations, resealed erythrocytes containing the active ingredient, and immunologically based formulations. The route(s) of administration will be readily apparent to the skilled artisan and will depend upon any number of factors including the type and severity of the disease being treated, the type and age of the veterinary or human subject being treated, and the like.

In some embodiments, a pharmaceutical composition of the present invention is formulated for local delivery via injection. In some embodiments, the pharmaceutical composition is designed for sustained release at the delivery site. In some embodiments, the composition comprises a microsphere, microparticle, nanosphere, nanoparticle, biodegradable gel, hydrogel, matrix, or polymer which releases at least one compound over time to the localized area. Examples of injectable liquid polymer compositions suitable for local delivery in methods of the invention can be found in the art (e.g., U.S. patent application Ser. No. 17/278,930).

Although the invention herein is principally directed to the ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist may design and perform such modification with merely ordinary, if any, experimentation. Subjects to which administration of the pharmaceutical compositions is contemplated include, but are not limited to, humans and other primates, mammals including commercially relevant mammals such as cattle, pigs, horses, sheep, cats, rodents, and dogs.

In one embodiment, the compositions utilized in the invention are formulated using at least one pharmaceutically acceptable excipient or carrier. In one embodiment, the pharmaceutical compositions comprise a therapeutically effective amount of a compound or conjugate of the invention and a pharmaceutically acceptable carrier. Pharmaceutically acceptable carriers that are useful, include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In one embodiment isotonic agents, for example, sugars, sodium chloride, or polyalcohols such as mannitol and sorbitol, are included in the composition. Prolonged absorption of the injectable compositions may be brought about by including in the composition an agent that delays absorption, for example, aluminum monostearate or gelatin. In one embodiment, the pharmaceutically acceptable carrier is not DMSO alone.

Formulations may be employed in admixtures with conventional excipients, i.e., pharmaceutically acceptable organic or inorganic carrier substances suitable for oral, vaginal, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

As used herein, “additional ingredients” include, but are not limited to, at least one of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” that may be included in the pharmaceutical compositions of the invention are known in the art and described, for example in Genaro, ed. (1985, Remington's Pharmaceutical Sciences, Mack Publishing Co., Easton, PA), which is incorporated herein by reference.

The composition utilized in the invention may comprise a preservative from about 0.005% to 2.0% by total weight of the composition. The preservative is used to prevent spoilage in the case of exposure to contaminants in the environment. Examples of preservatives useful in accordance with the invention included but are not limited to those selected from the group consisting of benzyl alcohol, sorbic acid, parabens, imidurea and combinations thereof. An exemplary preservative is a combination of about 0.5% to 2.0% benzyl alcohol and 0.05% to 0.5% sorbic acid.

In one embodiment, the composition includes an antioxidant and a chelating agent that inhibits the disassembly of the compound. Exemplary antioxidants for some compounds are BHT, BHA, alpha-tocopherol and ascorbic acid in the range of about 0.01% to 0.3%. In one embodiment, the BHT is in the range of 0.03% to 0.1% by weight by total weight of the composition. In one embodiment, the chelating agent is present in an amount of from 0.01% to 0.5% by weight by total weight of the composition. Exemplary chelating agents include edetate salts (e.g., disodium edetate) and citric acid in the weight range of about 0.01% to 0.20%. In one embodiment, chelating agents may be in the range of 0.02% to 0.10% by weight by total weight of the composition. The chelating agent is useful for chelating metal ions in the composition that may be detrimental to the shelf life of the formulation. While BHT and disodium edetate are the exemplary antioxidant and chelating agent respectively for some compounds, other suitable and equivalent antioxidants and chelating agents may be substituted therefore as would be known to those skilled in the art.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise at least one additional ingredient including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one which comprises a carbon-containing liquid molecule and which exhibits a less polar character than water. Liquid solutions of the pharmaceutical composition for use in the invention may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

In some embodiments, the liquid solution is a viscous gel. In some embodiments, the gel composition comprises at least one gelling agent. Examples of gelling agents include, but are not limited to, poloxamers, chitosan, methylcellulose, ethylcellulose, propylcellulose, carboxymethylcellulose, hydroxyethylcellulose, hydroxymethylcellulose, hydroxypropylcellulose, pectin, xylulose, Carbopol, guar gum, gellan gum, xanthan gum, gum acacia, pullulan, tragacanth, starch, carbomer, alginates, gelatin, polyvinylacohols, bentonite, carrageenan, hyaluronic acid, polyethylene oxide, polypropylene oxide, and polycarbophil. In some embodiments, the gel composition comprises at least one compound which accelerates gelation. Examples of acceptable compounds include, but are not limited to, calcium chloride, calcium bromide, calcium iodide, and calcium lactate.

In some embodiments, the composition is an injectable liquid polymer composition. Injectable liquid polymer compositions may comprise at least one biodegradable polymer. Examples of biodegradable polymers include, but are not limited to, polyglycolides, polylactides, polycaprolactones, polyanhydrides, polyorthoesters, polydioxanones, polyacetals, polyesteramides, polyamides, polyurethanes, polycarbonates, polyphosphazenes, polyketals, polyhydroxybutyrates, polyhydorxyvalerates, polyhyaluronic acid, polyalkylene oxalates, and polyalkylene oxides. In some embodiments, the injectable liquid polymer composition comprises at least one biocompatible solvent and/or co-solvent. Examples of biocompatible solvents include, but are not limited to, a-tocopherol, acetone, acetyl tributylcitrate, acetyl triethyl citrate, benzyl alcohol, butanol, butyrolactone, caprolactone, castor oil, n-cyclohexyl-2-pyrrolidone, diethylene glycol monomethyl ether, dimethyl acetamide, dimethyl formamide (DMF), dimethyl sulfoxide (DMSO), ethyl acetate, ethyl lactate, N-ethyl-2-pyrrolidone, glycerol, glycofulol, hydrogenated castor oil, isobutanol, isopropanol, N-hydroxyethyl-2-pyrrolidone, isopropylidene glycerol, lactic acid, lauric acid, laurate esters, smethoxypolyethylene glycol, methoxypropylene glycol, methyl acetate, methyl ethyl ketone, methyl lactate, N-methyl-2-pyrrolidone (NMP), oleic acid, oleate esters, polyethylated castor oil, polyethylated hydrogenated castor oil, low-molecular weight polyethylene glycol (PEG), low-molecular weight polysorbates (e.g., 20, 40, 60, or 80), propanol, propylene glycol, 2-pyrrolidone, sorbitan monolaurate, sorbitan monooleate, sorbitan monostearate, stearic acid, stearoyl esters, triacetin, tributyl citrate, and triethyl citrate. Examples of injectable liquid polymer compositions suitable for use in the invention can be found in the art (e.g., U.S. patent application Ser. No. 17/278,930).

Powdered and granular formulations of a pharmaceutical preparation of the composition utilized in the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise at least one of dispersing or wetting agent, a suspending agent, and a preservative. Additional excipients, such as fillers and sweetening, flavoring, or coloring agents, may also be included in these formulations.

A pharmaceutical composition for use in the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise at least one emulsifying agent such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

The regimen of administration may affect what constitutes an effective amount. The therapeutic formulations may be administered to the subject either prior to or after a diagnosis of disease. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a subject, such a mammal, including a human, may be carried out using known procedures, at dosages and for periods of time effective to prevent or treat disease. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the subject being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound for use in the invention is from about 1 and 5,000 mg/kg of body weight/per day. One of ordinary skill in the art would be able to study the relevant factors and make the determination regarding the effective amount of the therapeutic compound without undue experimentation.

The invention may be practiced as frequently as several times daily, or it may be practiced less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg/kg day dose may be initiated on Monday with a first subsequent 5 mg/kg per day dose administered on Wednesday, a second subsequent 5 mg/kg per day dose administered on Friday, and so on. The frequency of the dose will be readily apparent to the skilled artisan and will depend upon any number of factors, such as, but not limited to, the type and severity of the disease being treated, the type and age of the animal, etc.

Actual dosage levels of the active ingredients in the pharmaceutical compositions of this invention may be varied so as to obtain an amount of the active ingredient that is effective to achieve the desired therapeutic response for a particular subject, composition, and mode of administration, without being toxic to the subject.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a disease in a subject.

In certain embodiments, the composition of the present invention provides for a controlled release of a therapeutic agent. In certain instances, controlled-or sustained-release formulations of a pharmaceutical composition of the invention may be made using conventional technology, using for example proteins equipped with pH sensitive domains or protease-cleavable fragments. In some cases, the dosage forms to be used can be provided as slow or controlled-release of at least one active ingredient therein using, for example, hydropropylmethyl cellulose, other polymer matrices, gels, permeable membranes, osmotic systems, multilayer coatings, micro-particles, liposomes, or microspheres or a combination thereof to provide the desired release profile in varying proportions. Suitable controlled-release formulations known to those of ordinary skill in the art, including those described herein, can be readily selected for use with the pharmaceutical compositions of the invention. Thus, single unit dosage forms suitable for oral administration, such as tablets, capsules, gel-caps, lozenges, and caplets, which are adapted for controlled-release are encompassed by the present invention.

Most controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased subject compliance. In addition, controlled-release formulations can be used to affect the time of onset of action or other characteristics, such as blood level of the drug, and thus can affect the occurrence of side effects.

Most controlled-release formulations are designed to initially release an amount of drug that promptly produces the desired therapeutic effect, and gradually and continually release of other amounts of drug to maintain this level of therapeutic effect over an extended period of time. In certain embodiments, the controlled-release formulation of the composition described herein allows for release of a therapeutic agent precisely when the agent is most needed. In another embodiment, the controlled-release formulation of the composition described herein allows for release of a therapeutic agent precisely in conditions in which the therapeutic agent is most active. In order to maintain this constant level of drug in the body, the drug must be released from the dosage form at a rate that will replace the amount of drug being metabolized and excreted from the body.

In certain embodiments, the composition provides for an environment-dependent release, when and where the therapeutic agent is triggered for release. For example, in certain embodiments the composition invention releases at least one therapeutic agent when and where the at least one therapeutic agent is needed. The triggering of release may be accomplished by a variety of factors within the microenvironment of the treatment or prevention site, including, but not limited to, temperature, pH, the presence or activity of a specific molecule or biomolecule, and the like.

Controlled-release of an active ingredient can be stimulated by various inducers, for example pH, temperature, enzymes, water or other physiological conditions or compounds. The term “controlled-release component” in the context of the present invention is defined herein as a compound or compounds, including, but not limited to, polymers, polymer matrices, gels, permeable membranes, liposomes, or microspheres or a combination thereof that facilitates the controlled-release of the active ingredient.

In certain embodiments, the formulations of the present invention may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release that is longer that the same amount of agent administered in bolus form.

For sustained release, the compounds may be formulated with a suitable polymer or hydrophobic material that provides sustained release properties to the compounds. As such, the compounds for use the method of the invention may be administered in the form of microparticles, for example, by injection or in the form of wafers or discs by implantation.

In one embodiment of the invention, the compositions are administered to a subject, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

In one embodiment, the invention is practiced in dosages that range from one to five times per day or more. In another embodiment, the invention is practiced in range of dosages that include, but are not limited to, once every day, every two, days, every three days to once a week, and once every two weeks. It will be readily apparent to one skilled in the art that the frequency of administration of the various combination compositions of the invention will vary from subject to subject depending on many factors including, but not limited to, age, disease or disorder to be treated, gender, overall health, and other factors. Thus, the invention should not be construed to be limited to any particular dosage regime and the precise dosage and composition to be administered to any subject will be determined by the attending physical taking all other factors about the subject into account.

Routes of administration of include oral, nasal, rectal, parenteral, sublingual, transdermal, transmucosal (e.g., sublingual, lingual, (trans)buccal, (trans)urethral, vaginal (e.g., trans-and perivaginally), (intra)nasal, and (trans)rectal), intravesical, intrapulmonary, intraduodenal, intragastrical, intrathecal, subcutaneous, intramuscular, intradermal, intra-arterial, intravenous, intrabronchial, inhalation, and topical administration. In some embodiments, the route of administration is direct injection to the tissue in which the ciliopathy manifests.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### EXPERIMENTAL EXAMPLES

The following non-limiting Examples serve to illustrate selected embodiments of the invention. It will be appreciated that variations in proportions and alternatives in elements of the components shown will be apparent to those skilled in the art and are within the scope of embodiments of the present invention.

Without further description, it is believed that one of ordinary skill in the art can, using the preceding description and the following illustrative examples, make and utilize the present invention and practice the claimed methods. The following working examples therefore, point out specific embodiments of the present invention, and are not to be construed as limiting in any way the remainder of the disclosure.

### Example 1: A CRISPR Activation Screen Reveals a Cilia Disassembly Pathway Mutated in Focal Cortical Dysplasia

The materials and methods are described herein.

### Mammalian Cell Lines

NIH-3T3, RPE1-hTERT (RPE1), and HEK-293T parent cell lines were obtained; NIH-3T3 FlpIn cells were provided. Cells were cultured in a humidified 37° C. incubator with 5% CO2. RPE1 cells were passaged in DMEM/F-12 medium (Gibco) supplemented with 100 units/ml of Penicillin, 100 μg/ml Streptomycin, 2 mM Glutamine, and 10% fetal bovine serum (FBS, Gemini. NIH-3T3 and HEK-293T cells were passaged in DMEM, high glucose (Gibco) supplemented with 100 units/ml Penicillin, 100 μg/ml Streptomycin, 2 mM Glutamine, 1 mM sodium pyruvate (Gibco), and 10% FBS (Hyclone). Where indicated, NIH-3T3 cells were serum starved in 0.5% FBS-containing medium, and RPE1 cells were serum starved in 0.2% FBS-containing medium. Where specified, cells were treated with 250 nM Smoothened Agonist (SAG, Enzo Life Sciences), 6 μM Vorapaxar (VWR), 30 μM DSRM-3716 (Tocris), 10 μM dehydronitrosonisodipine (dHNN; Toronto Research Chemicals), 5 μM or 10 μM Dantrolene (as indicated, Tocris), 10 μM Y-27632,5 μM BAPTA-AM, 2 μM tubacin, 100 nM cytochalasin D (Sigma Aldrich), 15 μM lysophosphatidic acid (Sigma-Aldrich) 500 mM 3-acetylpyridine (Sigma Aldrich), 250 nM Torin1 (APExBIO), 10 U/ml bovine thrombin (Sigma Aldrich) or 1 μg/ml doxycycline (Fisher Scientific).

### Culture of Primary Mouse Cortical Neurons

For primary cortical neuron cultures, P0 pups from Balb/C mice were used. Prior to neuronal dissection, acid-treated 12 mm coverslips in a 24-well plate were coated with 20 μg/ml Poly-D-Lysine for 1 hour and then washed with dH₂O. Pups were anesthetized through hypothermia induction, followed by decapitation. Cortical tissue was dissected and isolated in ice-cold HBSS with no calcium or magnesium (Thermo Fisher). Cortical tissue was digested with 200 units of papain (Worthington) in HBSS with 4 mg of L-cysteine for 40 minutes. Cells were mechanically dissociated in Neurobasal media with a p1000 pipette and plated onto coverslips at a density of 300,000 cells per coverslip. Cells were initially incubated in medium containing Neurobasal plus (Thermo Fisher), 2% B27 plus supplement (Thermo Fisher), 1% penicillin/streptomycin (Thermo Fisher), and 10% heat-inactivated fetal bovine serum (FBS) (Sigma-Aldrich) for 3-6 hrs. Following incubation, media was changed into the same media without FBS. For small molecule treatments, DMSO or DSRM-3716 was added directly to media as specified. Coverslips were fixed with 4% PFA in PBS for 15 min prior to immunostaining (see below).

### DNA Cloning

CRISPRa sgRNAs were cloned by ligating annealed oligonucleotides into pXPR502 (Addgene #96923) digested with Esp3I (Thermo Fisher). CRISPR knockout sgRNAs were cloned by ligating annealed oligonucleotides into pMCB320 (Addgene #89359) digested with BstXI and Bpu110I (Thermo Fisher). Ligated products were transformed into chemically competent DH5a cells prepared in-house. Plasmid pXPR502-mCherry was cloned through PCR amplification of mCherry and insertion into pXPR502 to yield PP7-p65-HSF1-P2A-mCherry-T2A-PuroR.

PB-Tet-F2R-3xFlag was cloned by Gibson assembly of a synthesized mouse F2R gene product (Twist Biosciences) into a piggyBac Tet-Cas9 plasmid with Neo resistance. PB-Tet-SARM1-3xFlag was cloned through insertion of mouse SARM1(PCR amplified from pGW1-Myc-SARM1; Addgene #50707) into PB-Tet-F2R-3xFlag using traditional molecular biology. SARM1 point mutants were introduced into PB-Tet-SARM1-3xFlag by PCR and standard molecular biology (human V331E and G528S mutations were introduced in mouse SARM1; the amino acid number and identity of Val331 and Gly528 is the same for mouse and human SARM1 genes). PB-Tet-3xFlag-RhoA-P75S and RhoA-Q63L were cloned by Gibson assembly of synthesized 3xFlag-RhoA variants into PB-Tet-SARM1-3xFlag.

CRISPRa plasmid EF1α-dCas9-VP64-BFP was cloned by PCR of BFP and replacement of GFP in dCas9-VP64-GFP (Addgene #61422). For live-cell imaging, lentiviral constructs for labeling cilia (pCW-Crys-Htr6-3xmNeonGreen_Pgk-PA-mCherry-T2A-Blast) and centrioles (pCW-Pgk-miRFP670-Centrin2) were cloned by Gibson assembly from related plasmids used for cilia imaging (A K Ganga et al., 2021, Curr Biol 31: 2895-2905 e2897).

### Lentivirus Production

Lentiviral particles were produced by co-transfection of HEK293T cells with a lentiviral vector and packaging plasmids (pCMV-VSVG and pCMV-AR-8.91 for all constructs except for knockout sgRNAs in pMCB320, for which pCMV-VSVG, pRSV-Rev, and pMDLg/RRE were used). After transfection with polyethyleneimine (linear, MW ˜25,000, Polysciences), virus-containing supernatant was collected, filtered through a 0.45 μm polyethersulfone filter, and concentrated 3-fold with Lenti-X Concentrator (Clontech). For sgRNA libraries, lentiviral supernatants were not concentrated.

### Determination of Multiplicity of Infection

To assess multiplicity of infection, cells were seeded in a six-well plate and 24 hr later transduced with a series of dilutions of lentivirus-containing media with 4 μg/ml polybrene.

Transduced cells were subsequently split into duplicate wells of a 24-well plate; in one well, transduced cells were selected with 2 μg/ml puromycin while the other well was kept as an unselected control. After 72 hr, cell viability was measured by CellTiter-Blue (Promega) and quantified using a fluorescence plate reader (Molecular Devices SpectraMax iD3). Comparison of viability in puromycin-selected wells to control wells provided an estimate of transduction efficiency and multiplicity of infection.

### Production of Stable Cell Lines

NIH-3T3 and RPE1 cell lines were modified for expression of transgenes or sgRNAs by lentiviral transduction or piggy Bac-mediated transposition. Specifically, to generate the CRISPRa cell line used for genome-wide screening, dCas9-VP64-BFP was stably transduced into previously established NIH-3T3 cells containing a Hh-responsive blasticidin reporter (DK Breslow et al., 2018, Nat Genet 50: 460-471), following by sorting of a BFP-positive cell pool (Beckton Dickinson FACSAria III). To generate CRISPRa cell lines for select genes of interest, sgRNA constructs were stably transduced into the NIH-3T3 CRISPRa screening cell line described above. Briefly, cells were exposed to media with lentiviral particles diluted to an appropriate titer in medium containing 4 μg/ml polybrene (Sigma Aldrich). After 24 hr incubation, virus-containing medium was replaced with growth medium. After an additional 24 hr, cells were passaged, followed by selection for transduced cells by addition of 2 μg/ml puromycin (Invivogen). CRISPR knockout cell lines were prepared as described above except that NIH-3T3 cells expressing Cas9-BFP (DK Breslow et al., 2018, Nat Genet 50: 460-471) were transduced with lentiviral particles produced from pMCB320 sgRNA plasmids.

For stable introduction of doxycycline-inducible transgenes, the piggyBac system was used to introduce transposon cargos into NIH-3T3 FlpIn cells lacking dCas9 and the Hh reporter gene or into RPE1 cells modified to stably express Cas9-P2A-Blast (RPE1-Cas9) (AK Ganga et al., 2021, Curr Biol 31: 2895-2905 e2897). Transposition was achieved by co-transfection of a transposon vector and piggyBac transposase with Lipofectamine 2000 (Thermo Fisher) in NIH-3T3 cells or X-tremeGENE 9 (Roche) in RPE1 cells. After 48 hr incubation at 37° C., cells were passaged, followed by selection with 0.6-1 mg/ml G418 (Invivogen).

To generate cells for live imaging of cilia and centrioles, RPE1 cells were sequentially transduced with pHR-Pgk-Cas9-BFP (DK Breslow et al., 2018, Nat Genet 50: 460-471), pCW-Crys-Htr6-3xmNeonGreen_Pgk-PA-mCherry-T2A-Blast, and pCW-Pgk-miRFP670-Centrin2. A clonal line was isolated by FACS and fluorescent marker expression verified by microscopy.

### Analysis of Ciliation Rate

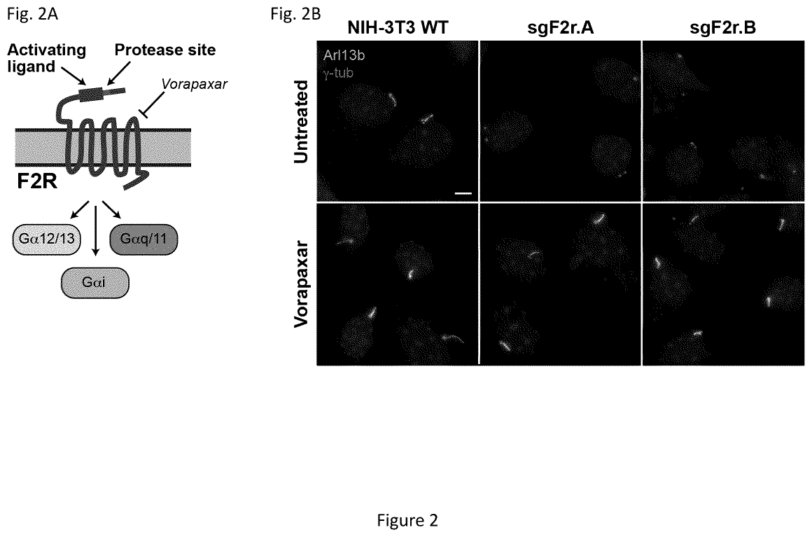

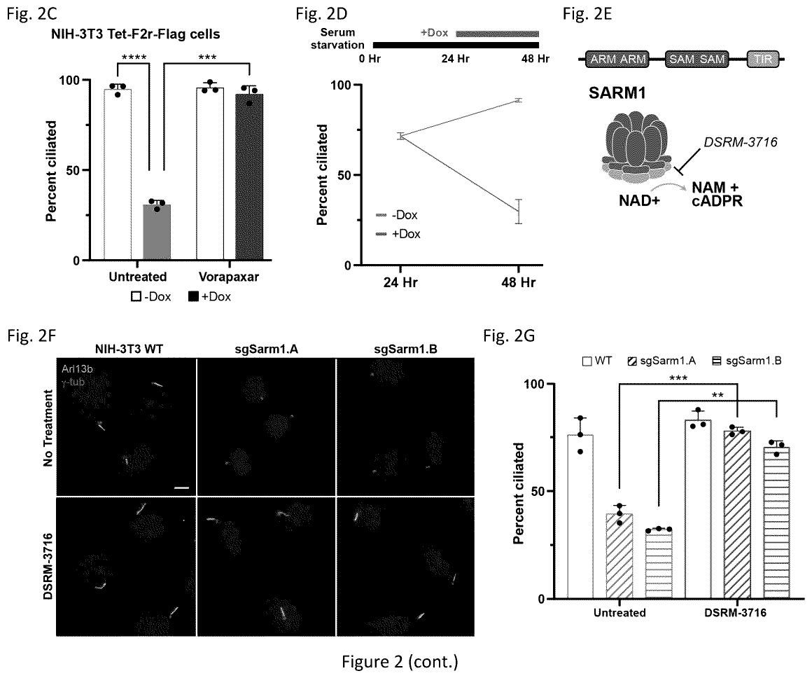

To analyze ciliation, NIH-3T3 cells were plated on acid-washed 13-mm round #1.5 coverslips additionally coated with poly-L-lysine; RPE1 cells were plated on uncoated, acid-washed 13 mm round #1.5 coverslips. To induce ciliogenesis by serum starvation, NIH-3T3 cells were transferred to 0.5% serum medium for 24 hr, and RPE1 cells were transferred to 0.2% serum medium for 48 hr. Where indicated, inhibitors were added at the time of serum starvation-apart from dantrolene, which was added to NIH-3T3 cells 24 hr prior to serum starvation and then continued through starvation. Where doxycycline-inducible transgenes were included, doxycycline was added at the time of serum starvation, except as noted in FIG. 2D.

To assess cilium assembly in proliferating NIH-3T3 cells that were not serum-starved, NIH-3T3 cells with Hh reporter and Cas9 (DK Breslow et al., 2018, Nat Genet 50: 460-471) were seeded on glass coverslips in media with 10% FBS and drug treatments were applied for 24 hr prior to fixation. To analyze serum-induced cilia disassembly, these cells were seeded on glass coverslips, serum starved for 24 hr, followed by addition of 10% FBS medium with or without indicated inhibitors for 24 hr. For analysis of ciliation in proliferating RPE1 cells, RPE1-Cas9 cells were seeded on glass coverslips in media with 10% FBS and cultured for 48 hr with or without drug treatments. To analyze LPA-induced cilia disassembly, RPE1-Cas9 cells were serum starved for 48 hr, followed by addition of 15 μM LPA in serum starvation medium for an additional 48 hr.

### Immunofluorescence Staining and Microscopy

Cells grown on coverslips were fixed in 4% paraformaldehyde, ice-cold methanol, or both in succession. Fixed coverslips were then blocked with PBS+3% bovine serum albumin+5% normal donkey serum, permeabilized with PBS+0.1% Triton X-100, incubated with the appropriate primary antibodies (Ar113b, Antibodies Inc. /NeuroMab 75-287; Cep164, Sigma Aldrich HPA037606; Acetylated Tubulin, Sigma Aldrich T6793; Gamma Tubulin, Sigma Aldrich T5326; Phospho-S6 Ribosomal Protein, Cell Signaling Technology 5364; CaMKII, Cell Signaling Technology 50049; Fop/Cep43, Abnova H00011116-M01) and secondary antibodies coupled to Alexa Fluor 488, Cy3, Cy5 (Jackson Immunoresearch) or CF-750 (Biotium), and stained with Hoechst DNA dye. Slides were then mounted with Fluoromount-G mounting medium (Electron Microscopy Sciences) and imaged using a Nikon Eclipse Ti-2 widefield microscope equipped with a CMOS camera (Photometrics Prime BSI), a 60× PlanApo oil objective (NA 1.40; Nikon Instruments), and an LED light source (Lumencor SOLA-V-NIR). Images were acquired at room temperature using Nikon Elements Software.

Widefield fluorescent microscopy images were analyzed using Fiji/ImageJ or Nikon Elements. Unless otherwise noted, quantification of ciliation was completed by manual counting of Arl13b-labeled cilia using centrosomal markers as a reference (where indicated, acetylated tubulin was used as an alternate ciliary marker). All images shown and used for quantification are maximum intensity z-projections, with the exception of phospho-S6 staining, which is shown for a single focal plane. To quantify cilia length, Nikon Elements software segment. AI module was used to identify cilia (following training using manually labeled reference images), and the GA3 analysis module to measure lengths of identified cilia.

Live-cell imaging was performed on a Nikon Eclipse Ti-2 microscope equipped with a Yokogawa W1 spinning disk unit, CMOS camera (Photometrics Prime BSI), 40× PlanApo silicone oil objective (NA 1.25), and 4-color laser combiner (Nikon Instruments LUN-F XL 405/488/561/640). Cells per well were seeded in glass-bottom dishes, serum starved for 48 hr, and imaged in phenol-red-free DMEM/F-12 medium following treatment with 10 U/ml thrombin. A Perfect Focus System was used to maintain focus, and a stage-top incubator (Tokai Hit) maintained 37° C. temperature and 5% CO₂.

### Genome-Wide Crispra Screening

For CRISPRa screening, the Caprano CRISPRa library (Addgene #92383 for Set A and #92384 for Set B) was used to target 22,774 mouse genes. The Set A and Set B portions of the sgRNA library each contain three sgRNAs per gene and were screened sequentially. For amplification of the Addgene-supplied library, plasmid DNA was transformed into NEBstable Competent E. coli (New England Biolabs) and extracted using Plasmid Plus Giga Kit (Qiagen). For each sgRNA set, lentiviral particles were produced as described above and transduced into dCas9-VP64 NIH3T3 reporter cells at a low multiplicity of infection (<0.4) while maintaining a >300:1 ratio of transduced cells to sgRNA library elements. Transduced cells were selected with puromycin for at least 5 days, and a ˜1000:1 ratio of cells to sgRNA elements was maintained during all subsequent steps. For Hh signaling and blasticidin selection, confluent cells were serum starved and treated with SAG for 24 hr, passaged to fresh plates and allowed to adhere, and then selected with 5 μg/ml blasticidin (or left unselected as a control). Cells were then passaged twice in blasticidin-free growth media before a second round of signaling and selection. Selected and unselected cell pools were then harvested for sgRNA analysis at the end of the experiment.

Genomic DNA was isolated from screen samples using the QIAamp DNA Blood Maxi Kit (Qiagen). Genomic DNA was then amplified with NEBNext High-Fidelity 2× PCR Master Mix (New England Biolabs) as previously described (KR Sanson et al., 2018, Nat Commun 9, 5416). Importantly, PCR primers amplified the sgRNA cassette while also introducing sample-specific barcodes, adapters for Illumina sequencing, and spacer nucleotides upstream of the protospacer to avoid synchronous readout of constant regions of the sgRNA cassette. PCR products were gel-purified, quantified on a Qubit 4 Fluorometer (Invitrogen) with the dsDNA BR Assay Kit (Thermo Scientific), and pooled for sequencing on a NovaSeq instrument (Illumina) at an average read depth of 1000 reads per sgRNA library element.

Sequencing data from the genome-wide screen was analyzed as previously described (DW Morgens et al., 2016, Nat Biotechnol 34: 634-636). Briefly, trimmed protospacer sequences were extracted from raw sequencing reads using BBduk software (Joint Genome Institute) and aligned to Caprano library reference sequences via the makeCounts. py script in the casTLE software package, which uses Bowtie to perform alignment with zero mismatches tolerated. The analyzeCounts. py script (v1.0) of casTLE was then used to calculate enrichment/depletion likelihood scores for each gene from the aggregate effects of all associated sgRNAs, as well as to estimate the phenotypic effect size (magnitude of enrichment/depletion). P values indicating the likelihood that similar results could be obtained from non-targeting control sgRNAs were determined using the addPermutations. py script with 100,000 permutations of non-targeting sgRNAs, yielding a minimum reported P value of 1×10⁻⁵.

### Rt-PCR Analysis of Gene Expression

For RT-qPCR analysis, RNA was extracted with Direct-zol RNA Miniprep Kit (Zymo Research) and reverse transcribed using High-Capacity cDNA Reverse Transcription Kit (Applied Biosystems). SYBR FAST qPCR master mix (Kapa Biosystems) was used for quantitative PCR in triplicate reactions carried out in a QuantStudio 6 Flex Real-Time PCR System (Life Technologies). RNA expression levels were calculated using the ddCt method relative to GAPDH, using the average from three technical replicates for each biological replicate.

### Analysis of Blasticidin Sensitivity in Hh Reporter Cells

To quantify blasticidin resistance conferred by Hh reporter gene expression, NIH-3T3 cells were serum starved for 24 hr with or without SAG treatment, passaged to 24-well plates, and then cultured at a range of blasticidin concentrations. After 4 days of selection, cell viability was measured using the CellTiter-Blue kit (Promega) and a fluorescence plate reader (Molecular Devices SpectraMax iD3). Measurements of cell viability were normalized such that growth in the absence of blasticidin represented 100% growth.

For growth competitions using Gli3-mCherry reporter cells, sgGli3-mCherry cells were co-cultured with wildtype (mCherry-negative) reporter cells at a ratio of ˜30:70. Stimulation of signaling via SAG treatment and selection with 5 μg/ml blasticidin was carried out as described above. The fraction of mCherry-positive cells in unselected and selected populations was determined by flow cytometry (Beckton Dickinson Fortessa X-20) and analysis with Flow-Jo software.

### Statistical Analyses

To assess overlap of cilia disassembly pathway genes and FMCD genes, Fisher's exact test was used to assess an association between these gene properties. To assess statistical significance in genome-wide CRISPRa screening, the maximum-likelihood casTLE method (20) was used, and random permutations of control non-targeting sgRNAs were used to determine statistical significance. To compare cilia lengths in neurons and in thrombin-treated cells, the Kolmogorov-Smirnov test was applied to data aggregated from N=3 replicate experiments. All other statistical tests were based on t-test (two-sided, unpaired, unequal variance with Welch's correction). The number of replicate experiments, definitions of center and dispersion, and other statistical test details can be found in the Figure Legends. P values for t-tests and violin plots were generated using GraphPad Prism software.

The results are described herein.

### Crispra Screen for Negative Regulators of Ciliary Signaling

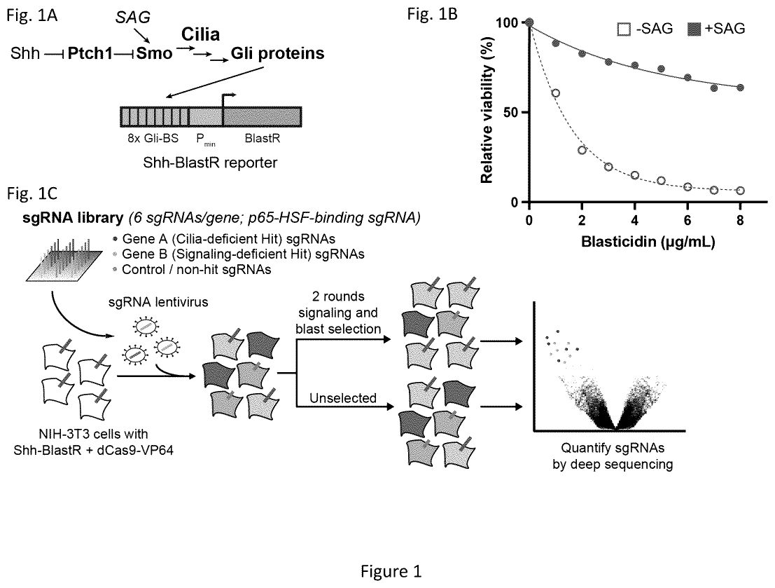

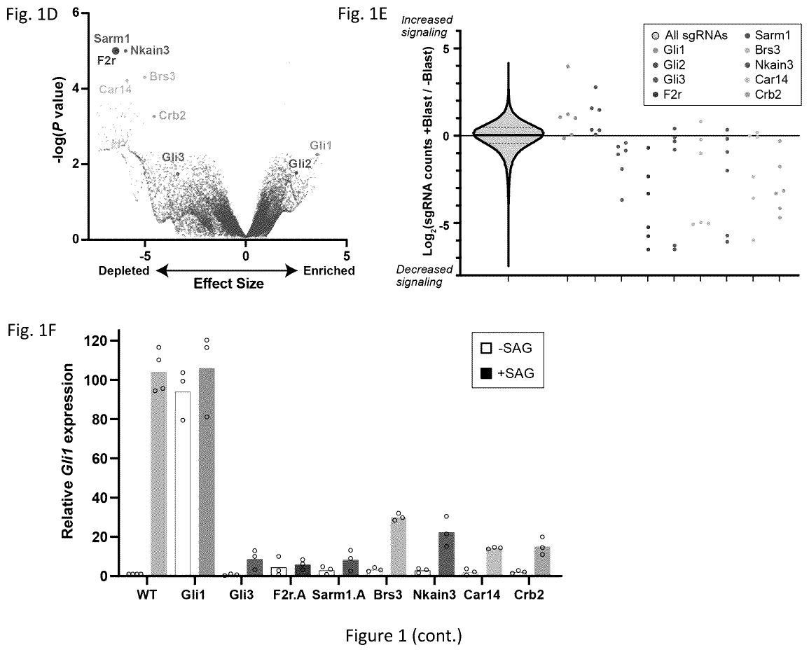

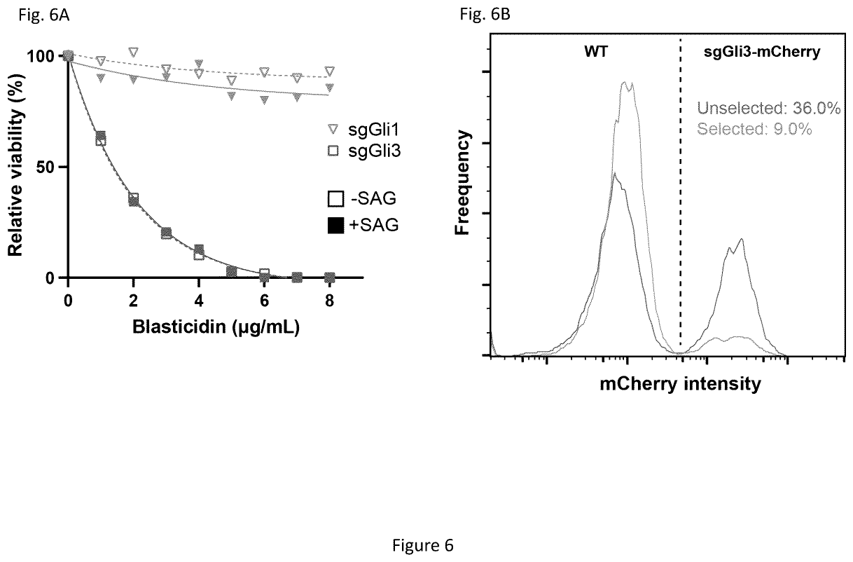

Genome-scale loss-of-function screens have been used to systematically identify genes that mediate cilium assembly, many of which are mutated in congenital ciliopathies (J Kim et al., 2010, Nature 464: 1048-1051; G Wheway et al., 2015, Nat Cell Biol 17: 1074-1087; S Roosing et al., 2015, Elife 4: e06602; G V Pusapati et al., 2018, Dev Cell 44: 113-129; D K Breslow et al., 2018, Nat Genet 50: 460-471). It was thus reasoned that a gain-of-function screen would provide a complementary means to uncover negative regulators of cilia function, including genes that control cilia disassembly. To achieve this goal, mouse NIH-3T3 fibroblasts were engineered to combine CRISPRa-based gene overexpression (KR Sanson et al., 2018, Nat Commun 9, 5416) with a previously established Hh pathway transcriptional reporter (DK Breslow et al., 2018, Nat Genet 50: 460-471). In brief, this reporter translates Hh signaling-dependent activation of Gli transcription factors-a process that is strictly dependent on primary cilia (F Bangs et al., 2017, Cold Spring Harb Perspect Biol 9)-into expression of a blasticidin resistance gene (FIG. 1A). To functionally validate this cell line, the effect of Hh pathway stimulation on blasticidin resistance was assessed in the wildtype reporter cell line versus reporter cells transduced with sgRNAs targeting Gli1 and Gli3, which activate and repress Hh signaling, respectively. As expected, stimulation of Hh signaling with SAG, a Smo agonist, led to a strong increase in blasticidin resistance in wildtype reporter cells (FIG. 1B). In Glil CRISPRa cells, blasticidin resistance was observed even without Hh pathway stimulation, whereas in Gli3 CRISPRa cells, SAG-induced blasticidin resistance was diminished (FIG. 6A). Furthermore, in a growth competition experiment, flow cytometry revealed that Gli3 CRISPRa cells (marked by mCherry) became strongly depleted upon blasticidin selection relative to parental cells (FIG. 6B). Thus, the reporter cell line effectively translates Hh pathway output into blasticidin resistance and allows identification of CRISPRa perturbations that modulate cilium-dependent Hh signaling.

For a pooled genome-wide screen, a screening strategy was applied in which a library of CRISPRa sgRNAs is transduced into reporter cells, followed by two rounds of SAG-mediated stimulation of Hh signaling, with and without application of blasticidin selection (FIG. 1C). The Caprano CRISPRa sgRNA library, which targets >22,000 mouse genes with six guides per gene, was used (K R Sanson et al., 2018, Nat Commun 9, 5416). After signaling and selection, deep sequencing was used to quantify sgRNA abundances in selected versus unselected cells (FIG. 1C). To identify genes that affect ciliary signaling, a statistical framework termed castle (DW Morgens et al., 2016, Nat Biotechnol 34: 634-636) was used to estimate phenotypic effect sizes as well as P values for each gene.

Examining screen results, it was noted that many hit genes were depleted upon blasticidin selection (inhibited Hh signaling), including the repressive Hh effector Gli3 (FIG. 1D). A smaller number of hit genes became enriched by potentiating SAG-induced Hh signaling, including the activating Hh effectors Gli1 and Gli2. Overall, 30 genes were hits with P<0.001, and 267 were hits at P<0.01. For many hit genes, multiple sgRNAs were strongly depleted or enriched, as expected for bona fide hits (FIG. 1E). To validate the results, cell lines expressing individual sgRNAs targeting Gli1, Gli3, and several hit genes not previously linked to cilia function or Hh signaling were made. These latter genes include: F2R, a protease-activated GPCR (A Chandrabalan et al., 2021, FEBS J 288: 2697-2726); SARM1, an NADase regulator of neurodegeneration (M D Figley et al., 2020, Curr Opin Neurobiol 63: 59-66); Brs3, a GPCR linked to obesity (H Ohki-Hamazaki et al., 1997, Nature 390: 165-169); Crb2, a cell polarity gene linked to brain and kidney defects (A Slavotinek et al., 2015, Am J Hum Genet 96: 162-169); Nkain3, and Car14. For each of these CRISPRa cell lines, Hh signaling was assessed by RT-PCR analysis of Hh target gene Gli1, a readout that is distinct from the blasticidin reporter gene used in the initial screen. Notably, all hits tested showed expected alterations in Glil expression (FIG. 1F). The CRISPRa gain-of-function screen provides a complementary resource that identifies distinct and novel ciliary regulators. While some hits from the CRISPRa screen exhibit limited overlap with those identified in a CRISPR KO screen with the same reporter (DK Breslow et al., 2018, Nat Genet 50: 460-471) or with the 686 ciliary genes in the SysCilia database (many of which were identified from loss-of-function approaches) (S S V Vasquez et al., 2021, Mol Biol Cell 32: br13), many were newly identified. Collectively, these data indicate that the screen effectively identified known and novel genes whose expression modulates ciliary Hh signaling.

### F2R GPCR and SARM1 NADase Induce Cilium Disassembly

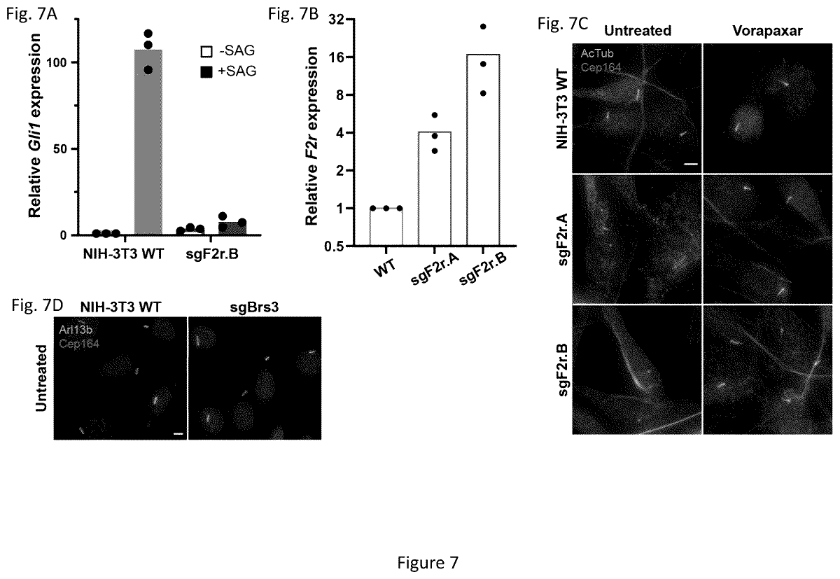

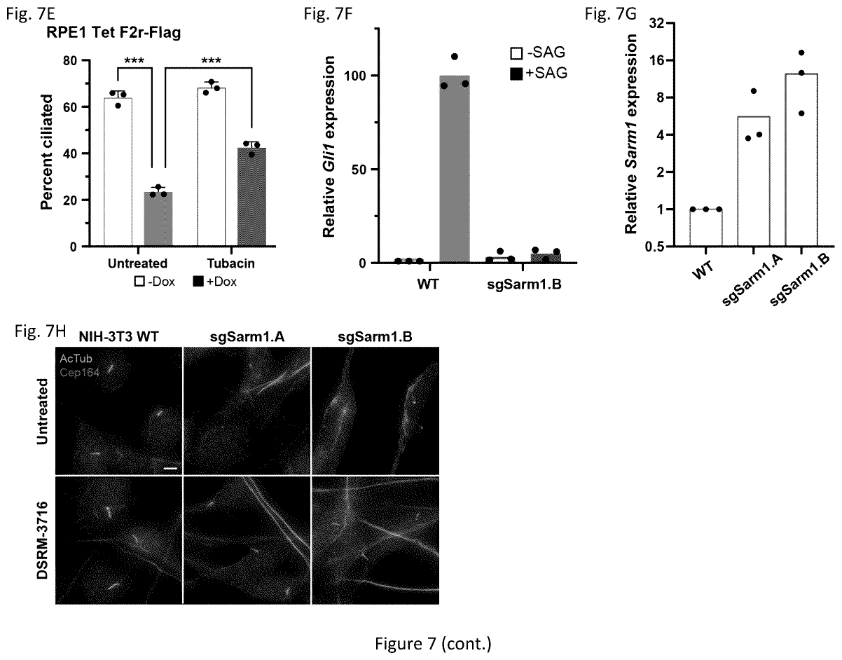

Coagulation factor II thrombin receptor (F2R; Protease-activated receptor 1 [Par1]) is a GPCR that regulates wound healing, neural tube closure, and platelet activation (E Camerer et al., 2010, Dev Cell 18: 25-38; MN Adams et al., 2011, Pharmacol Ther 130: 248-282). Proteolytic cleavage of F2R by thrombin or other proteases exposes an N-terminal activation domain that triggers signaling through Gaq/11, Ga12/13, and Gai/o (FIG. 2A) (A Chandrabalan et al., 2021, FEBS J 288 :2697-2726). To understand how F2R CRISPRa impairs ciliary signaling, NIH-3T3 cell lines expressing two F2R-targeting sgRNAs from the screen (sgF2R. A, sgF2R. B) were made, which both increased F2R expression and inhibited Hh signaling (FIG. 1F, FIG. 7A, and FIG. 7B). In these cells, a significant decrease in ciliation was observed after ciliogenesis was induced by serum starvation for 24 hr (FIG. 2B). This phenotype was fully rescued upon treatment with the F2R inhibitor Vorapaxar (S Chackalamannil et al., 2008, J Med Chem 51: 3061-3064), thus establishing the specificity of the F2R CRISPRa phenotype (FIG. 2B). Similar results were obtained using acetylated tubulin as a ciliary marker, confirming that F2R impairs ciliation (FIG. 7C). It was noted that cilia loss was seen for F2R, but not in cells overexpressing Brs3, a second GPCR that was a hit in the screen. In Brs3 CRISPRa cells, Hh signaling is defective while ciliation is normal (FIG. 7D), thus indicating that screen hits impact ciliary signaling by diverse mechanisms.

To test if F2R induces cilia disassembly (versus represses cilia assembly), a Doxycycline (Dox)-inducible F2R-Flag construct was used for temporal control of F2R overexpression. As for F2R CRISPRa, this transgene reduces ciliation when induced prior to ciliogenesis, an effect that was again rescued by Vorapaxar (FIG. 2C). The experiment was then modified such that cells were first allowed to ciliate, followed by treatment with or without Dox for a further 24 hr. In untreated control cells, high ciliogenesis was observed through the experiment. By contrast, Dox treatment induced a marked decrease in ciliation, indicating that F2R can provoke disassembly of previously formed cilia (FIG. 2D). To test the generality of this F2R activity, the Tet-F2R-Flag construct was inserted into RPE1-hTERT (RPE1) cells, a human cell line widely used to study cilia dynamics. As in NIH-3T3 cells, Dox-treated RPE1 Tet-F2R-Flag cells exhibited significantly reduced ciliation (FIG. 7E). It was also noted that F2R-Flag was not observed to localize to cilia. Lastly, it was determined if F2R-induced cilia disassembly occurs through a similar mechanism as when quiescent cells are stimulated with serum mitogens.

Because tubulin deacetylase HDAC6 is a key component of this pathway (I Sanchez et al., 2016, Nat Cell Biol 18: 711-717), RPE Tet-F2R-Flag cells were treated with the HDAC6 inhibitor tubacin and observed significant rescue of F2R-induced cilia disassembly (FIG. 7E). Together, these results establish F2R as a novel GPCR mediator of cilia disassembly in mouse and human cells.

Screen hit SARM1 (Sterile alpha and TIR motif-containing 1) is an octameric NADase found on mitochondria and in the cytoplasm that cleaves NAD+ to yield nicotinamide and cyclic-ADP-ribose (cADPR) (FIG. 2E) (K Essuman et al., 2017, Neuron 93: 1334-1343 e1335; J Gerdts et al., 2015, Science 348: 453-457; J Gerdts et al., 2013, J Neurosci 33: 13569-13580).

Recently, SARM1 has recently emerged as a key mediator of axonal and neuronal degeneration (J Gerdts et al., 2015, Science 348: 453-457; J M Osterloh et al., 2012, Science 337: 481-484; Y Li et al., 2022, J Cell Biol 221; T J Waller, 2022, Front Cell Neurosci 16: 958900). Genetic inactivation of SARM1 is protective in models of neurodegeneration and retinal degeneration, including syndromes that arise from ciliary defects (e.g., Leber's Congenital Amaurosis and Retinitis Pigmentosa) (M D Figley et al., 2020, Curr Opin Neurobiol 63: 59-66 ; E Ozaki et al., 2020, Life Sci Alliance 3; L Gibbons et al., 2022, Front Neurosci 16: 852114; Y Sasaki et al., 2020, Elife 9). To investigate how SARM1 CRISPRa disrupts ciliary signaling, NIH-3T3 cell lines expressing two SARM1-targeting sgRNAs (sgSARM1.A, sgSARM1.B) were made, and impaired Hh signaling and SARM1 overexpression by RT-PCR were confirmed (FIG. 1F, FIG. 7F, and FIG. 7G). After inducing cilia formation by serum starvation, a significant decrease in ciliation was observed for sgSARM1.A and sgSARM1.B CRISPRa cells (FIG. 2F and FIG. 2G). To confirm the specificity of this phenotype, DSRM-3716, a validated inhibitor of SARM1 NADase activity, was used (RO Hughes et al., 2021, Cell Rep 34: 108588). Notably, DSRM-3716 fully suppressed cilia loss seen in SARM1 CRISPRa cells (FIG. 2F, FIG. 2G, and FIG. 7H). Thus, screen hits F2R and SARM1 both induce cilia loss when overexpressed.

### F2R Disassembles Cilia Through SARM1 and Ryanodine Receptor-Mediated Calcium Release

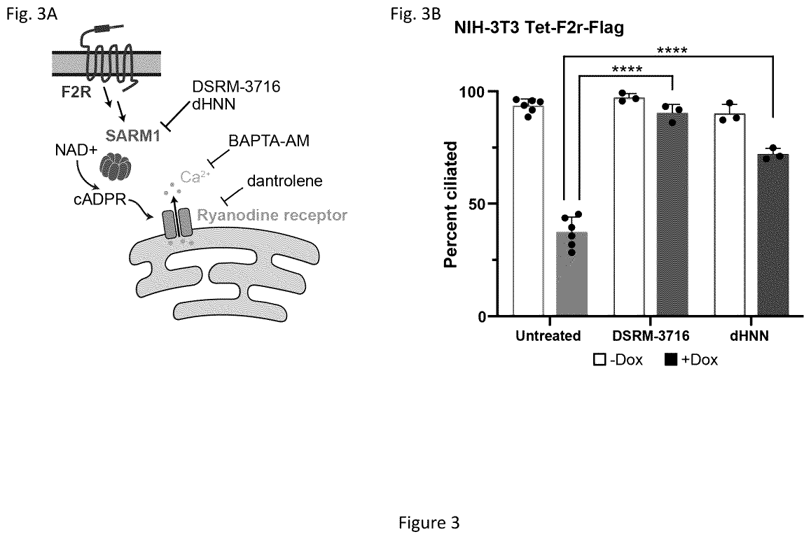

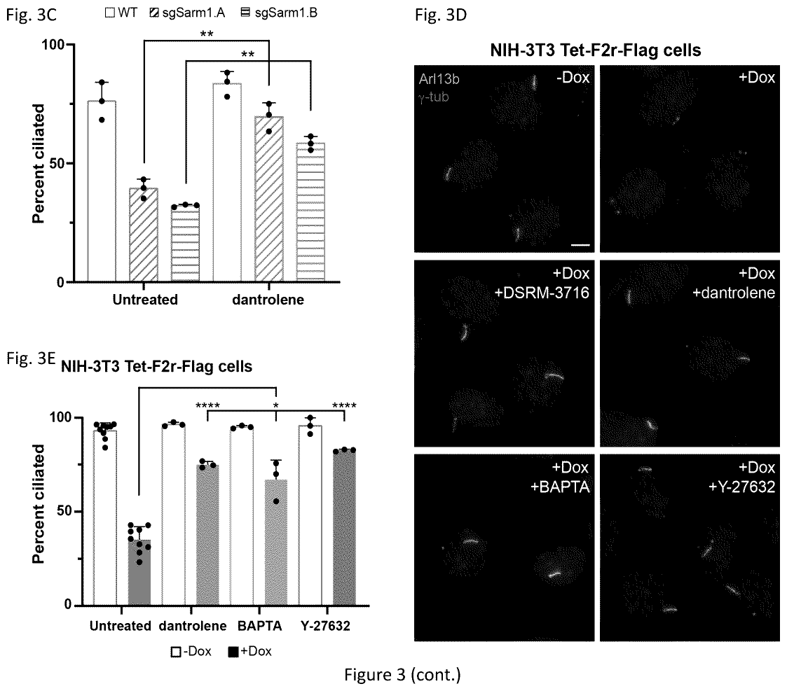

Given that F2R is a GPCR and SARM1 is an intracellular enzyme and that both disrupt ciliation when overexpressed, they may act together in a shared pathway, with SARM1 serving as a downstream mediator of cilia disassembly initiated by F2R (FIG. 3A). To test this, Tet-F2R NIH-3T3 cells were treated with SARM1 inhibitor DSRM-3716 and a strong rescue of ciliation was observed (FIG. 3B, FIG. 3D). Similar results were observed with a second SARM1 inhibitor, dHNN (W H Li et al., 2021, Elife 10), further supporting the model that SARM1 is an intracellular effector of cilia disassembly initiated by F2R. Given SARM1's established role in promoting axonal degeneration (J Gerdts et al., 2015, Science 348: 453-457; J M Osterloh et al., 2012, Science 337: 481-484; Y Li et al., 2022, J Cell Biol 221; T J Waller, 2022, Front Cell Neurosci 16: 958900), it is contemplated that there are shared mechanistic features of SARM1-mediated regulation of ciliary versus axonal microtubule-based structures.

To investigate the mechanism of cilia disassembly downstream of SARM1, it was examined whether cADPR is not only a product of SARM1 activity (Y Sasaki et al., 2020, Exp Neurol 329: 113252) but also a signaling molecule that stimulates Ryanodine Receptor (RyR) ion channels to release calcium from the ER (FIG. 3A) (A Galione et al., 1991, Science 253: 1143-1146; O A Ogunbayo et al., 2011, J Biol Chem 286: 9136-9140; R Coronado et al., 1994, Am J Physiol 266: C1485-1504). Calcium has long been thought to play a central and conserved role in cilia disassembly (E N Pugacheva et al., 2007, Cell 129: 1351-1363; M Mirvis et al., 2019, Plos Biol 17: e3000381; O V Plotnikova et al., 2012, Molecular Biology of the Cell 23: 2658-2670; R W Tucker et al., 1983, J Submicrosc Cytol 15: 139-143; L M Quarmby et al., 1994, J Cell Biol 124: 807-815), but the mechanism of calcium mobilization has eluded characterization. It was then tested whether SARM1 mediates cilia disassembly through RyR channel activation. Consistent with this model, the RyR inhibitor dantrolene (R Coronado et al., 1994, Am J Physiol 266: C1485-1504) conferred strong rescue of ciliation in cells overexpressing SARM1 or F2R (FIG. 3C-FIG. 3E). Further supporting RyR-mediated calcium release as a trigger for cilia disassembly, the cell-permeable calcium chelator BAPTA-AM similarly blocked F2R-induced cilia loss (FIG. 3D and FIG. 3E).

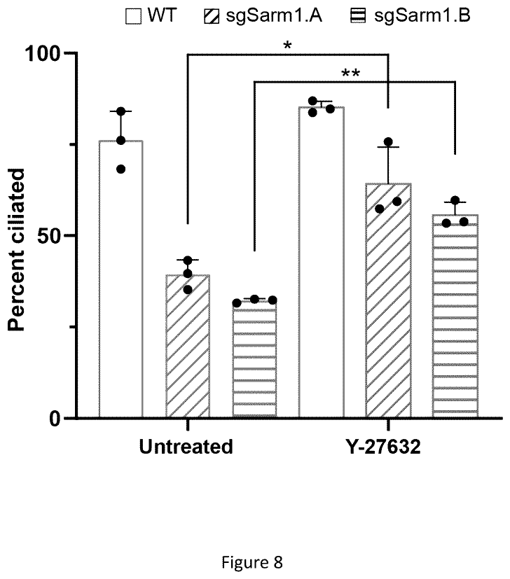

Calcium activates RhoA in a variety of contexts (S Sakurada et al., 2003, Circ Res 93: 548-556; S Varadarajan et al., 2022, J Cell Biol 221; H A Benink et al., 2005, J Cell Biol 168: 429-439; C Pardo-Pastor et al., 2018, Proc Natl Acad Sci U S A 115: 1925-1930; H Murakoshi et al., 2011, Nature 472: 100-104), and RhoA is an established target of F2R signaling (D L Greenberg et al., 2003, Biochemistry 42: 702-709; C B Martin et al., 2001, Oncogene 20: 1953-1963) implicated in regulation of cilia dynamics (A V R Lake et al., 2020, Biorxiv, 2020.2011.2026.393801; J Kim et al., 2015, Nat Commun 6: 6781; A Pitaval et al., 2010, J Cell Biol 191: 303-312; C E Jewett et al., 2021, Dev Cell 56: 325-340). It was thus tested if RhoA and Rho-activated Rock kinases contribute to the cilia disassembly observed. In support of this model, Rock inhibitor Y-27632 significantly rescued cilia disassembly mediated by F2R or SARM1 overexpression (FIG. 3D, FIG. 3E, and FIG. 8A). Together, these data indicate that F2R triggers cilia disassembly via SARM1-mediated cADPR production, RyR channel activation, release of ER calcium stores, and RhoA-activated Rock kinases.

### F2R and a SARM1-Directed Pathway are Key Endogenous Regulators of Cilia Dynamics

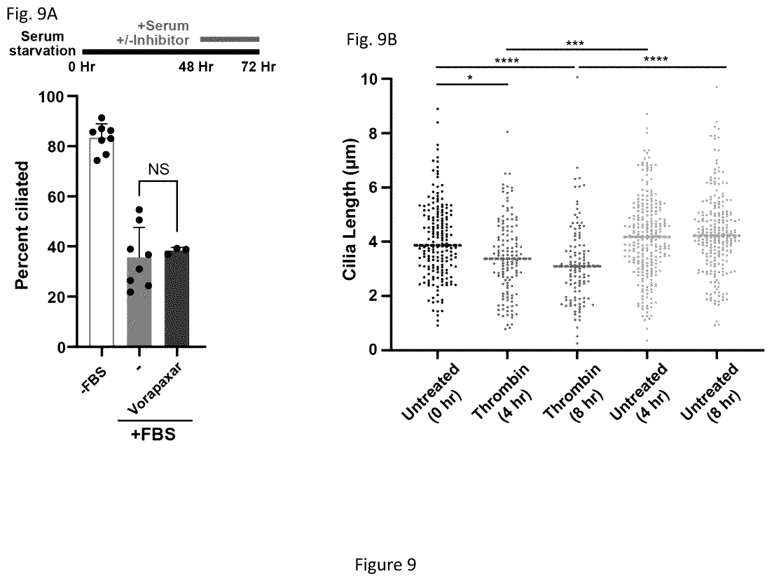

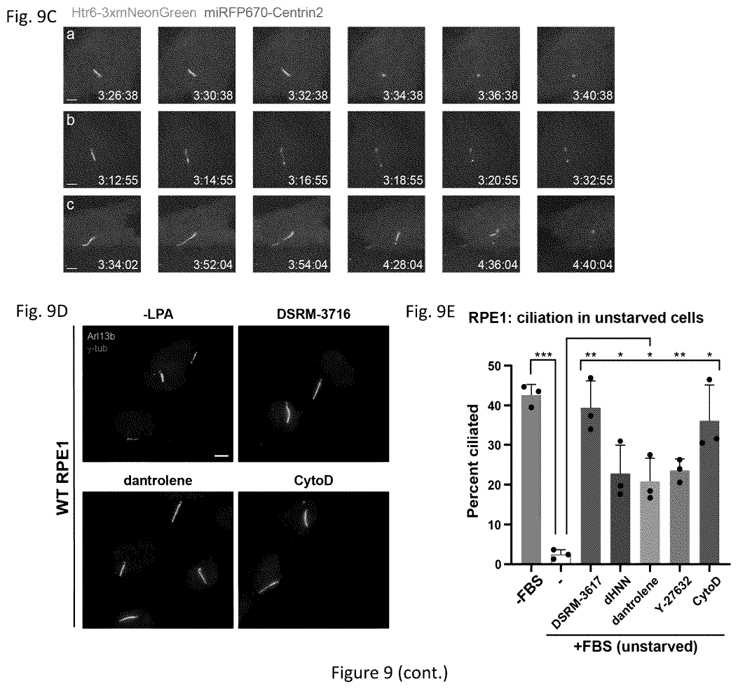

Having established that F2R and SARM1 induce a cilia disassembly pathway when overexpressed, it was then determined if this pathway also contributes to endogenous regulation of cilia dynamics. A key paradigm of regulated cilia disassembly is the long-standing observation that cilia of quiescent cells are resorbed when cell cycle re-entry is triggered by serum mitogens (I Sanchez et al., 2016, Nat Cell Biol 18: 711-717; R W Tucker et al., 1979, Cell 18: 1065-1072). As expected, robust cilia disassembly was observed when quiescent NIH-3T3 cells were stimulated with serum. However, when wildtype cells were treated with inhibitors of SARM1, RyR channels, or Rock kinases, cilia disassembly was potently blocked (FIG. 4A). This effect was similar in magnitude to that seen when cells were treated with Cytochalasin D (CytoD), an established inhibitor of cilia disassembly (J Kim et al., 2010, Nature 464: 1048-1051; CEL Smith et al., 2020, Front Cell Dev Biol 8: 622822; M Mirvis et al., 2018, Biochem J 475: 2329-2353). By contrast, F2R inhibitor Vorapaxar did not inhibit serum-induced cilia resorption, consistent with serum mitogens and F2R serving as distinct triggers of a shared cilia disassembly pathway (FIG. 9A).

Recently, lysophosphatidic acid (LPA) signaling through the LPAR1 GPCR has been identified as the main mediator of serum-induced cilia disassembly (V Walia et al., 2019, Dev Cell 50: 229-246; H B Hu et al., 2021, Nat Commun 12: 662). It was then determined if SARM1 and downstream components are needed for LPA-induced cilia disassembly. Here, the generality of the results was tested by using RPE1 cells, a widely used model of mitogen-induced cilia disassembly. As above, LPA-induced cilia disassembly was blocked when cells were treated with inhibitors of SARM1, RyR channels, or ROCK kinases (FIG. 4B).

It was then examined whether endogenously expressed F2R could trigger cilia disassembly upon activation by its cognate activator, thrombin. Notably, in serum-starved wildtype RPE1 cells, marked disassembly of cilia occurred following thrombin treatment (FIG. 4C and FIG. 4D). This disassembly was rapid and characterized by a progressive decrease in cilia length and prevalence (FIG. 4D and FIG. 9B). Live-cell imaging enabled direct visualization of cilia disassembly in response to thrombin treatment, including ciliary shedding and shortening events similar to those observed when cilia disassembly is triggered by serum mitogens (FIG. 4E and FIG. 9C) (M Mirvis et al., 2019, Plos Biol 17: e3000381). Furthermore, thrombin-induced cilia disassembly was potently blocked by vorapaxar and DSRM-3716, indicating that thrombin acts through F2R and SARM1 (FIG. 4C). Collectively, these data demonstrate that endogenous F2R and LPAR1 initiate a common pathway that requires SARM1, RyRs, and Rho/ROCK to induce cilia disassembly.

Some perturbations that inhibit cilia disassembly also increase cilia length and allow cilia formation under otherwise non-permissive conditions (C E L Smith et al., 2020, Front Cell Dev Biol 8: 622822; M Mirvis et al., 2018, Biochem J 475: 2329-2353; I Sanchez et al., 2016, Nat Cell Biol 18: 711-717). In support of a close relationship among these phenotypes, it was noted that the block in LPA-induced cilia disassembly observed above was accompanied by a significant increase in cilia length (FIG. 4F and FIG. 9D). Similarly, inhibition of SARM1, RyR channels, or Rock kinases enabled robust ciliation in non-serum-starved NIH-3T3 and RPE1 cells that typically do not form cilia (FIG. 4G and FIG. 9E). These effects were similar to those seen for CytoD, a potent modulator of cilia dynamics (J Kim et al., 2010, Nature 464: 1048-1051; CEL Smith et al., 2020, Front Cell Dev Biol 8: 622822; M Mirvis et al., 2018, Biochem J 475: 2329-2353). The roles for F2R and SARM1 in this process, has new and unexpected input on key functions in development, wound healing, and neurodegeneration (A Chandrabalan et al., 2021, FEBS J 288: 2697-2726; T J Waller, 2022, Front Cell Neurosci 16: 958900). Given the identification of thrombin/F2R signaling as a new physiologic cue that modulates cilia dynamics, it is noteworthy that loss of F2R function can cause exencephaly, a phenotype commonly generated by excess Hh signaling (E Camerer et al., 2010, Dev Cell 18: 25-38; J N Murdoch et al., 2010, Birth Defects Res A Clin Mol Teratol 88: 633-652).

### Cilia Disassembly Pathway Genes Are Mutated in Focal Cortical Dysplasia

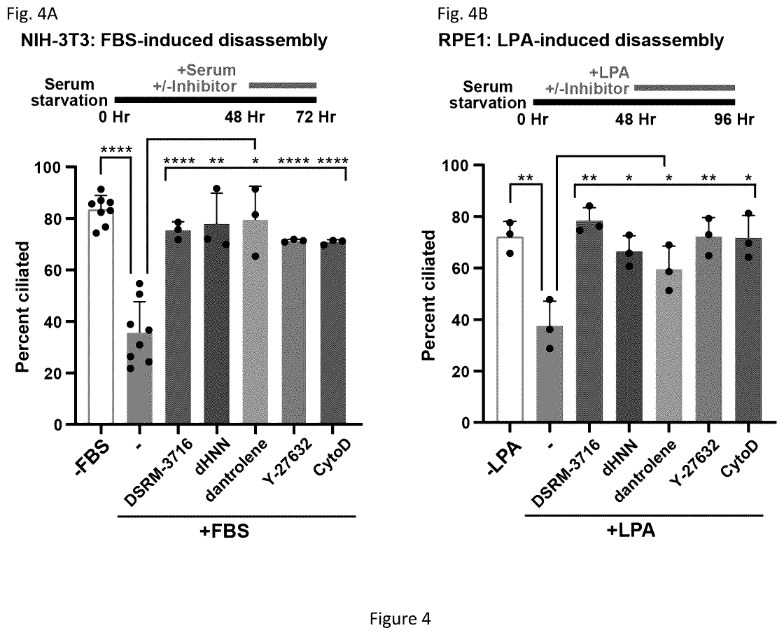

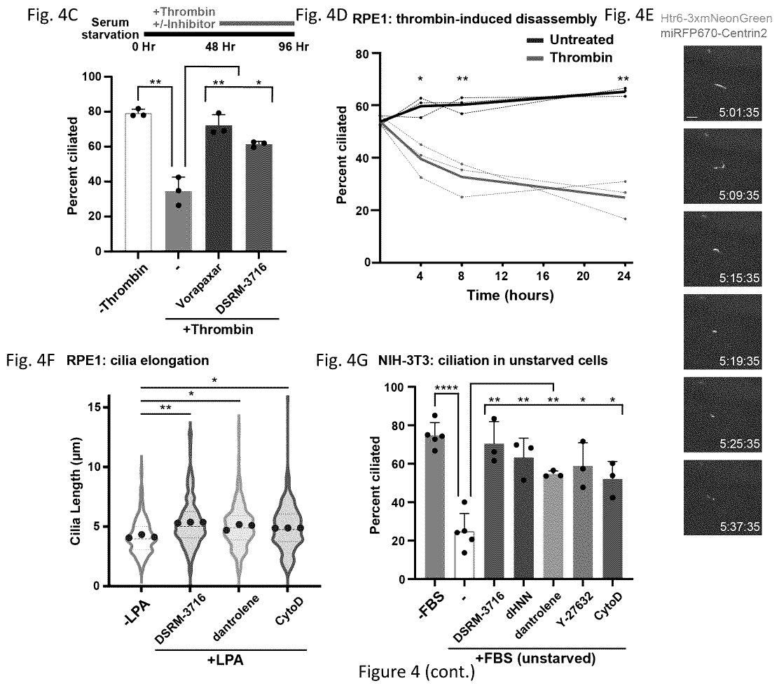

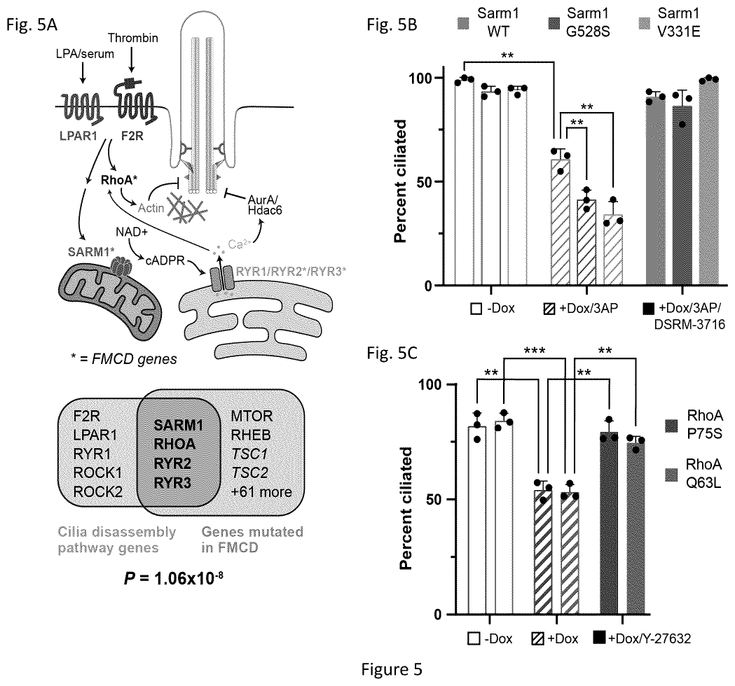

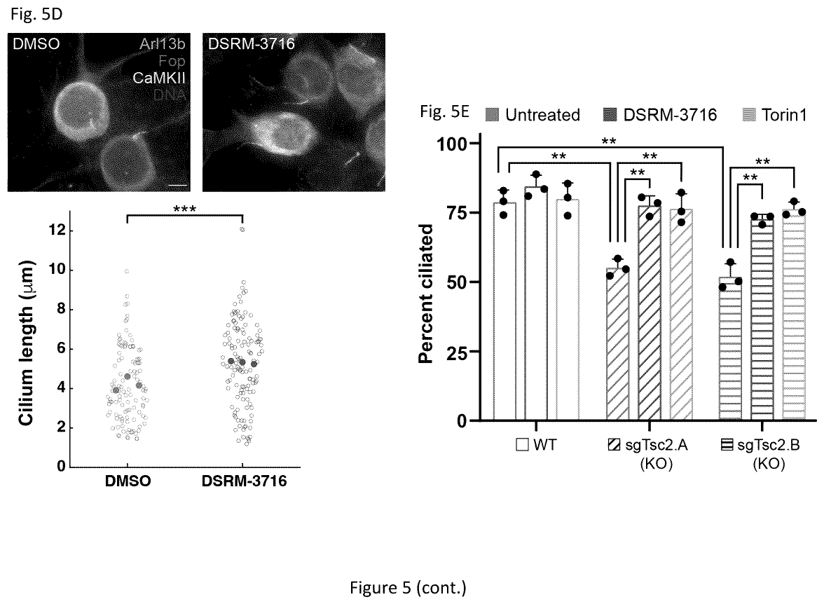

Having defined a novel cilia disassembly pathway, the functional consequences of altered pathway activity were then determined. In this context, a striking overlap between pathway components and those recently found to be mutated in patients with focal cortical dysplasia (FCD), a neurodevelopmental disorder that is part of the broader class of focal malformation of cortical development (FMCD) disorders, was noted (FIG. 5A) (C Chung et al., 2023, Nat Genet 55: 209-220). FMCD disorders include FCD, Tuberous Sclerosis Complex (TSC), and hemimegalencephaly and are typically caused by somatic mutations that locally impair cortical neuron patterning and function, often resulting in intractable epilepsy (P H Iffland et al., 2017, Annu Rev Pathol 12: 547-571). The observation that SARM1, RYR2, RYR3, and RHOA are all among 69 recently defined FMCD genes further supports a shared function for these genes (C Chung et al., 2023, Nat Genet 55: 209-220). Indeed, the association between FMCD genes and the cilia disassembly pathway identified here is highly significant (P=1.06×10⁻⁸, Fisher's exact test; FIG. 5A).

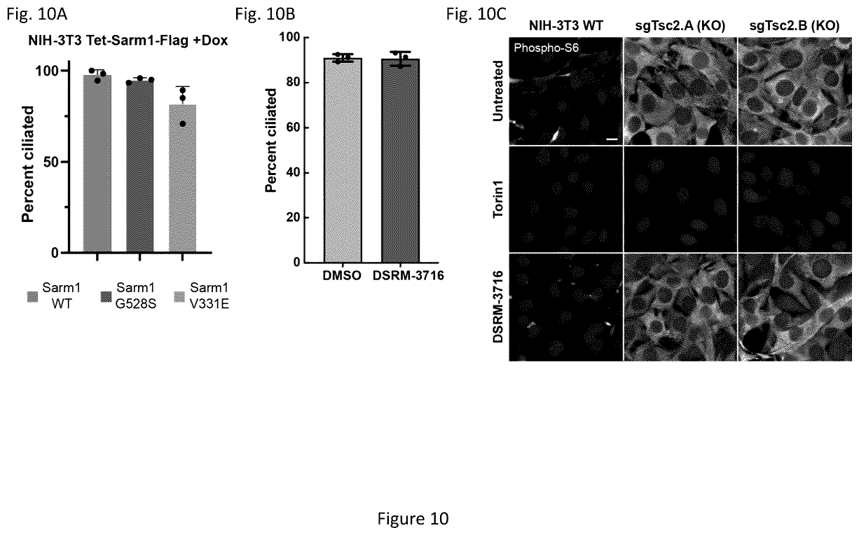

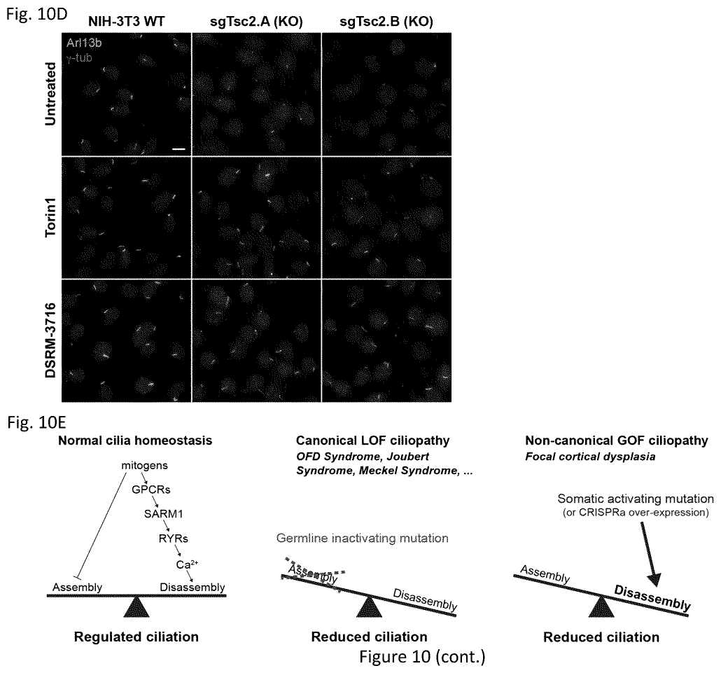

It was then determined if FMCD-associated variants in cilia disassembly pathway genes can impact cilia homeostasis. Specifically, because FMCD disorders commonly arise from somatic gain-of-function mutations (C Chung et al., 2023, Nat Genet 55: 209-220; P H Iffland et al., 2017, Annu Rev Pathol 12: 547-571; S Baldassari et al., 2019, Acta Neuropathol 138: 885-900; S Bizzotto et al., 2022, Nat Rev Neurosci 23: 275-286), it was determined if FMCD-associated mutations can cause aberrant loss of cilia, similar to that seen upon CRISPRa-based overexpression of disassembly mediators. An FCD-associated SARM1 variant that produces a G528S mutation was first evaluated (C Chung et al., 2023, Nat Genet 55: 209-220; AJ Bloom et al., 2022, Mol Neurodegener 17: 1). Tet-SARM1-Flag NIH-3T3 cell lines that inducibly overexpress wildtype SARM1, SARM1-G528S, or SARM1-V331E, a mutant previously shown to increase SARM1 activity, were established (J Gilley et al., 2021, Elife 10). For these transgenes, brief Dox-inducible expression and treatment with 3-acetylpyridine (3AP), an allosteric activator of SARM1, led to significantly reduced ciliation (FIG. 5B and FIG. 10A) (T Wu et al., 2021, Cell Rep 37: 109872). Notably, under these conditions the G528S and V331E SARM1 mutants both caused a significantly greater loss of cilia than wildtype SARM1 (FIG. 5B). These effects were fully reversed by DSRM-3716, confirming that SARM1-G528S reduces ciliation via its NADase activity. Thus, cilia loss is potentiated by an FCD-associated SARM1 variant, and this effect is phenocopied by an established hyperactive SARM1 mutant.

An FCD-associated RHOA-P75S mutation was then examined (C Chung et al., 2023, Nat Genet 55: 209-220). As for SARM1-G528S, induction of Tet-Flag-RhoA-P75S induced cilia loss in a manner similar to the known hyperactive mutant RhoA-Q63L (FIG. 5C). These effects were reversed by the Rock inhibitor Y-27632. Thus, patient-derived pathologic variants in the SARM1/RyR/RhoA pathway promote cilia disassembly.

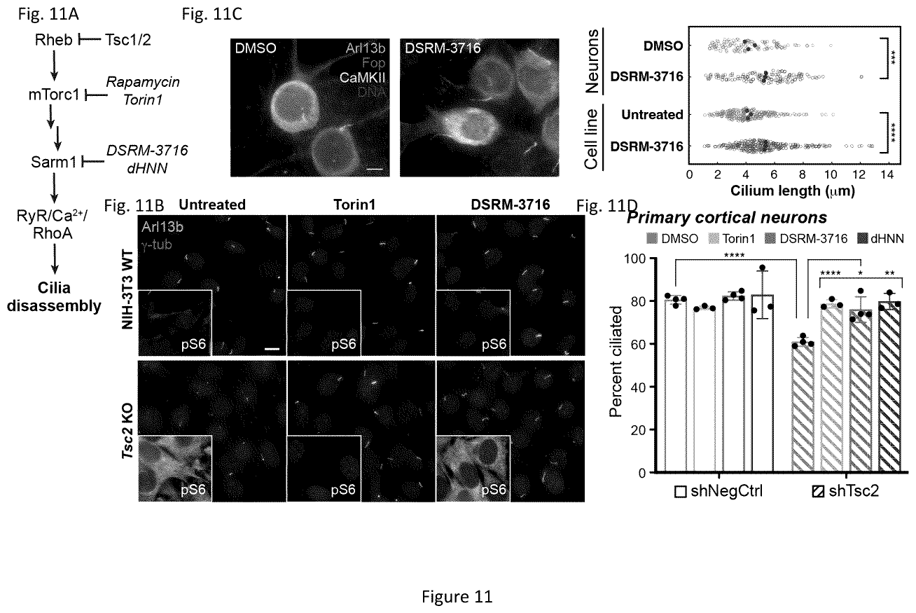

It was then examined if SARM1 regulates cilia dynamics in cortical neurons whose dysfunction underlies FCD. In primary cortical neurons from wildtype mice, robust ciliation was observed, with ˜90% of excitatory neurons possessing a readily detectable cilium (FIG. 10B, FIG. 11B, and FIG. 11D). Notably, in these neurons, the SARM1 inhibitor DSRM-3716 induced a significant increase in cilia length (FIG. 5D, FIG. 11C). This effect mirrors the observation of cilia elongation in RPE1 cells upon DSRM-3716 treatment (FIG. 4F and FIG. 9D) and indicates that SARM1's role in regulating ciliary dynamics is conserved in the neurons that drive FCD pathogenesis.

Lastly, in light of the overlap observed between the cilia disassembly pathway and genes mutated in FCD, it was then determined if other FCD genes can impact cilia dynamics. As FCD is most frequently caused by somatic mutations that increase mTORC1 pathway activity (C Chung et al., 2023, Nat Genet 55: 209-220; P H Iffland et al., 2017, Annu Rev Pathol 12:547-571; S Baldassari et al., 2019, Acta Neuropathol 138: 885-900; A Gerasimenko et al., 2023, Neurobiol Dis 182: 106144), the possibility that mTORC1-activating mutations might cause cilia loss was determined. While studies have reached conflicting conclusions regarding how mTORC1 influences cilia (T R Hartman et al., 2009, Hum Mol Genet 18: 151-163; T Rosengren et al., 2018, Cell Mol Life Sci 75: 2663-2680; A Di Nardo et al., 2020, Cell Rep 31: 107780), two reports find that mTORC1-activating mutations can cause cilia loss in cell culture and in FMCD patient neurons (P H Iffland et al., 2017, Annu Rev Pathol 12: 547-571; A Gerasimenko et al., 2023, Neurobiol Dis 182: 106144; S M Park et al., 2018, Neuron 99: 83-97). Consistent with this observation, the key mTORC1 negative regular Tsc2 was a hit in the CRISPR knockout (KO) screen previously conducted using the same Hh signaling reporter (D K Breslow et al., 2018, Nat Genet 50: 460-471). Thus, Tsc2 inactivation, as occurs in FCD and TSC (P H Iffland et al., 2017, Annu Rev Pathol 12: 547-571; J S Lim et al., 2017, Am J Hum Genet 100: 454-472), may impair Hh signaling by provoking mTORC1-driven cilia loss. To test this possibility, Tsc2 KO NIH-3T3 cells were generated using two different sgRNAs. As expected, mTORC1 hyperactivation was readily observed in Tsc2 KO cells via phospho-S6 staining (FIG. 10C) (X M Ma et al., 2009, Nat Rev Mol Cell Biol 10: 307-318). Ciliation was significantly impaired in Tsc2 KO cells, and this defect could be restored by the mTORC1 inhibitor Torin1 (FIG. 5E and FIG. 10D). Strikingly, strong rescue of ciliation was observed when Tsc2 KO cells were treated with SARM1 inhibitor DSRM-3716 (FIG. 5E and FIG. 10D). Thus, mTORC1-induced cilia loss occurs through SARM1-mediated disassembly. Consistent with SARM1 acting downstream of mTORC1, elevated phospho-S6 in Tsc2 KO cells was reversed by Torin1 but not by DSRM-3716 (FIG. 10C). Taken together, these findings indicate that aberrant cilia disassembly is a shared feature of multiple FCD-causing mutations and that associated ciliary defects may be corrected by SARM1 inhibition. That diverse FCD genes impair cilia function suggests that FCD can be considered as a non-canonical class of ciliopathy. In this model (FIG. 10E), pathologic cilia loss in FCD is caused by somatic activating mutations in cilia disassembly genes, forming a complement to canonical ciliopathies in which cilia loss is due to inactivating mutations in cilium assembly genes.

Systematic functional screening has proven a powerful approach to study primary cilia but has thus far been largely restricted to loss-of-function approaches. While CRISPRa screening has the potential to model non-physiologic overexpression conditions, the SARM1-mediated disassembly pathway is also endogenously required for cilia disassembly. Additionally, CRISPRa-derived overexpression phenotypes can provide insight into the functional impact of naturally occurring somatic mutations. Advances in human genetics are revealing the role of somatic mutations in a growing range of cells, processes, and disorders (S Olafsson et al., 2021, Trends Genet 37: 872-881; S Bizzotto et al., 2022, Nat Rev Neurosci 23: 275-286). Although CRISPRa may not always phenocopy somatic mutational effects, the CRISPRa screen enabled identification of a pathway activated by somatic mutation in FCD. Thus, similar gain-of-function screens using other cell types and readouts may reveal the underpinnings of additional pathologies driven by somatic mutations.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of treating or preventing a ciliopathy in a subject in need thereof comprising administering to the subject a composition comprising at least one inhibitor of proteins involved in cilia disassembly.

2. The method of claim 1, wherein the ciliopathy is a focal malformation of cortical development (FMCD) disorder.

3. The method of claim 2, wherein the FMCD disorder is focal cortical dysplasia (FCD).

4. The method of claim 1, wherein the at least one protein involved in cilia disassembly is selected from the group consisting of coagulation factor II receptor (F2R), sterile alpha and TIR motif containing 1 (SARM1), ryanodine receptor 1 (RyR1), ryanodine receptor 2 (RyR2), ryanodine receptor 3 (RyR3), Ras homolog member A (RhoA), Rho-associated protein kinase 1 (ROCK1), and Rho-associated protein kinase 2 (ROCK2).

5. The method of claim 4, wherein the at least one inhibitor of F2R is at least one selected from the group consisting of vorapaxar, atopaxar, SCH-79797, analogs, derivatives, and prodrugs thereof, and pharmaceutically acceptable salts and hydrates thereof.

6. The method of claim 4, wherein the at least one inhibitor of SARM1 is at least one selected from the group consisting of DSRM-3716, dehydronitrosonisodipine (dHNN), MY-9B, WX-02-37, EV-99, WX-02-34, WX-02-35, 3-oxo-5-((2-(trifulromethyl)phenyl)amino)-2,3-dihydroisothiazole-4-carbonitrile, 5-((2-chlorophenyl)amino)-3-oxo-2,3-dihydroisothiazole-4-carbonitrile, 5-((2-methylsulfonyl)phenyl)amino)-3-oxo-2,3-dihydroisothiazole-4-carbonitrile, 5-((6-chloroquinolin-5-yl)amino)-2-methyl-3-oxo-2,3-dihydroisothiazole-4-carbonitrile, 5-((6-methoxy-2-methylquinolin-5-yl)amino)-2-methyl-3-oxo-2,3-dihydroisothiazole-4-carbonitrile, nicotinic acid mononucleotide (NMN), 2-(4-Pyridinyl)-N-(2,2,2-trifluoroethyl)-1-pyrrolidinecarboxamide, 1-(Pyridin-4-ylmethyl)-3-(2,2,2-trifluoroethyl)urea, (S)-1-Methyl-1-(1-(pyridin-4- yl)ethyl)-3-(2,2,2-trifluoroethyl)urea, (R)-1-methyl-1-(1-(pyridin-4-yl)ethyl)-3-(2,2,2-trifluoroethyl)urea, (S)-1-methyl-1-(1-phenylethyl)-3-(2,2,2-trifluoroethyl)urea, 3-(3-chlorophenyl)-N-(4-methyl-3-(pyridin-4-yl)-1H-pyrazol-5-yl)propenamide, 3-(4-Chlorophenyl)-N-(4-methyl-3-(pyridin-4-yl)-1H-pyrazol-5-yl)propenamide, analogs, derivatives, and prodrugs thereof, and pharmaceutically acceptable salts and hydrates thereof.

7. The method of claim 4, wherein the at least one inhibitor of RyR1, RyR2, or RyR3 is selected from the group consisting of dantrolene, 6,7-(methylenedioxy)-1-octyl-4-quinolone-3-carboxylic acid, analogs, derivatives, and prodrugs thereof, and pharmaceutically acceptable salts and hydrates thereof.

8-9. (canceled)

10. The method of claim 4, wherein the at least one inhibitor of ROCK1 or ROCK2 is selected from the group consisting of thiazovivin, Y-27632, DJ4, GSK269962A, WAY-624704, RKI-1447, Chroman 1, azaindole 1, ATI13148, GSK429286A, Y-39983, ripasudil, fasudil, hydroxyfasudil, netarsudil, ZINC00881524, H-1152, belumosudil, analogs, derivatives, and prodrugs thereof, and pharmaceutically acceptable salts and hydrates thereof.

11. (canceled)

12. The method of any one of claim 4, wherein the method further comprises administering to the subject at least one selected from the group consisting of an inhibitor of lysophosphatidic acid receptor 1 (LPAR1), an inhibitor of mammalian target of rapamycin complex 1 (mTORC1), and a Ca²⁺ chelator.

13-14. (canceled)

15. The method of claim 12, wherein the at least one Ca²⁺ chelator is at least one selected from the group consisting of 1,2-bis(2-aminophenoxy)ethane-N,N,N′,N′-tetraacetic acid tetrakis(acetoxymethyl ester) (BAPTA-AM), ethylenediaminetetraacetic acid (EDTA), ethyleneglycoltetraacetic acid (EGTA), diethylenetriaminepentaacetate (DTPA), hydroxyethylethylenediaminetriacetic acid (HEEDTA), diaminocyclohexanetetraacetic acid (CDRA), 1,2-bis(2-aminophenoxy)ethane-N,N,N′,N′-tetraacetic acid (BAPTA), analogs, derivatives, and prodrugs thereof, and pharmaceutically acceptable salts and hydrates thereof.

16. A method of inhibiting cilia disassembly in a cell comprising contacting the cell with at least one inhibitor of at least one protein selected from the group consisting of F2R, SARM1, RyR1, RyR2, RyR3, RhoA, ROCK1, and ROCK2.

17. The method of claim 16, wherein the at least one inhibitor of F2R is at least one selected from the group consisting of vorapaxar, atopaxar, SCH-79797, analogs, derivatives, and prodrugs thereof, and pharmaceutically acceptable salts and hydrates thereof.

18. The method of claim 16, wherein the at least one inhibitor of SARM1 is at least one selected from the group consisting of DSRM-3716, dehydronitrosonisodipine (dHNN), MY-9B, WX-02-37, EV-99, WX-02-34, WX-02-35, 3-oxo-5-((2-(trifulromethyl)phenyl)amino)-2,3-dihydroisothiazole-4-carbonitrile, 5-((2-chlorophenyl)amino)-3-oxo-2,3-dihydroisothiazole-4-carbonitrile, 5-((2-methylsulfonyl)phenyl)amino)-3-oxo-2,3-dihydroisothiazole-4-carbonitrile, 5-((6-chloroquinolin-5-yl)amino)-2-methyl-3-oxo-2,3-dihydroisothiazole-4-carbonitrile, 5-((6-methoxy-2-methylquinolin-5-yl)amino)-2-methyl-3-oxo-2,3-dihydroisothiazole-4-carbonitrile, nicotinic acid mononucleotide (NMN), 2-(4-Pyridinyl)-N-(2,2,2-trifluoroethyl)-1-pyrrolidinecarboxamide, 1-(Pyridin-4-ylmethyl)-3-(2,2,2-trifluoroethyl)urea, (S)-1-Methyl-1-(1-(pyridin-4- yl)ethyl)-3-(2,2,2-trifluoroethyl)urea, (R)-1-methyl-1-(1-(pyridin-4-yl)ethyl)-3-(2,2,2-trifluoroethyl)urea, (S)-1-methyl-1-(1-phenylethyl)-3-(2,2,2-trifluoroethyl)urea, 3-(3-chlorophenyl)-N-(4-methyl-3-(pyridin-4-yl)-1H-pyrazol-5-yl)propenamide, 3-(4-Chlorophenyl)-N-(4-methyl-3-(pyridin-4-yl)-1H-pyrazol-5-yl)propenamide, analogs, derivatives, and prodrugs thereof, and pharmaceutically acceptable salts and hydrates thereof.

19. The method of claim 16, wherein the at least one inhibitor of RyR1, RyR2, or RyR3 is selected from the group consisting of dantrolene, 6,7-(methylenedioxy)-1-octyl-4-quinolone-3-carboxylic acid, analogs, derivatives, and prodrugs thereof, and pharmaceutically acceptable salts and hydrates thereof.

20-21. (canceled)

22. The method of claim 16, wherein the at least one inhibitor of ROCK1 or ROCK2 is selected from the group consisting of thiazovivin, Y-27632, DJ4, GSK269962A, WAY-624704, RKI-1447, Chroman 1, azaindole 1, ATI13148, GSK429286A, Y-39983, ripasudil, fasudil, hydroxyfasudil, netarsudil, ZINC00881524, H-1152, belumosudil, analogs, derivatives, and prodrugs thereof, and pharmaceutically acceptable salts and hydrates thereof.

23. (canceled)

24. The method of any one of claim 16, wherein the method further comprises contacting the cell with at least one selected from the group consisting of an inhibitor of LPAR1, an inhibitor of mTORC1, and a Ca²⁺ chelator.

25-26. (canceled)

27. The method of claim 24, wherein the at least one Ca²⁺ chelator is at least one selected from the group consisting of 1,2-bis(2-aminophenoxy)ethane-N,N,N′,N′-tetraacetic acid tetrakis(acetoxymethyl ester) (BAPTA-AM), ethylenediaminetetraacetic acid (EDTA), ethyleneglycoltetraacetic acid (EGTA), diethylenetriaminepentaacetate (DTPA), hydroxyethylethylenediaminetriacetic acid (HEEDTA), diaminocyclohexanetetraacetic acid (CDRA), 1,2-bis(2-aminophenoxy)ethane-N,N,N′,N′-tetraacetic acid (BAPTA), analogs, derivatives, and prodrugs thereof, and pharmaceutically acceptable salts and hydrates thereof.

28. A composition comprising:
a) at least one inhibitor of at least one protein selected from the group consisting of F2R, SARM1, RyR1, RyR2, RyR3, RhoA, ROCK1, and ROCK2; and
b) at least one selected from the group consisting of:
(i) at least one inhibitor of LPAR1;
(ii) at least one inhibitor of mTORC1; and
(iii) at least one Ca²⁺ chelator.

29. The composition of claim 28, wherein the at least one inhibitor of at least one protein selected from the group consisting of F2R, SARM1, RyR1, RyR2, RyR3, RhoA, ROCK1, and ROCK2 is at least one selected from the group consisting of vorapaxar, atopaxar, SCH-79797, argatroban, bivalirudin, dabigatran, lepirudin, desirudin, dantrolene, 6,7-(methylenedioxy)-1-octyl-4-quinolone-3-carboxylic acid, NSC 23766, zoledronic acid, EHT 1864, CCF-1423, ML141, thiazovivin, Y-27632, DJ4, GSK269962A, WAY-624704, RKI-1447, Chroman 1, azaindole 1, ATI13148, GSK429286A, Y-39983, ripasudil, hydroxyfasudil, netarsudil, ZINC00881524, H-1152, belumosudil, DSRM-3716, dehydronitrosonisodipine (dHNN), MY-9B, WX-02-37, EV-99, WX-02-34, WX-02-35, 3-oxo-5-((2-(trifulromethyl)phenyl)amino)-2,3-dihydroisothiazole-4-carbonitrile, 5-((2-chlorophenyl)amino)-3-oxo-2,3-dihydroisothiazole-4-carbonitrile, 5-((2-methylsulfonyl)phenyl)amino)-3-oxo-2,3-dihydroisothiazole-4-carbonitrile, 5-((6-chloroquinolin-5-yl)amino)-2-methyl-3-oxo-2,3-dihydroisothiazole-4-carbonitrile, 5-((6-methoxy-2-methylquinolin-5-yl)amino)-2-methyl-3-oxo-2,3-dihydroisothiazole-4-carbonitrile, nicotinic acid mononucleotide (NMN), 2-(4-Pyridinyl)-N-(2,2,2-trifluoroethyl)-1-pyrrolidinecarboxamide, 1-(Pyridin-4-ylmethyl)-3-(2,2,2-trifluoroethyl)urea, (S)-1-Methyl-1-(1-(pyridin-4-yl)ethyl)-3-(2,2,2-trifluoroethyl)urea, (R)-1-methyl-1-(1-(pyridin-4-yl)ethyl)-3-(2,2,2-trifluoroethyl)urea, (S)-1-methyl-1-(1-phenylethyl)-3-(2,2,2-trifluoroethyl)urea, 3-(3-chlorophenyl)-N-(4-methyl-3-(pyridin-4-yl)-1H-pyrazol-5-yl)propenamide, 3-(4-Chlorophenyl)-N-(4-methyl-3-(pyridin-4-yl)-1H-pyrazol-5-yl)propenamide, analogs, derivatives, and prodrugs thereof, and pharmaceutically acceptable salts and hydrates thereof.

30. The composition of claim 28, wherein the at least one Ca²⁺ chelator is at least one selected from the group consisting of 1,2-bis(2-aminophenoxy)ethane-N,N,N′,N′-tetraacetic acid tetrakis(acetoxymethyl ester) (BAPTA-AM), ethylenediaminetetraacetic acid (EDTA), ethyleneglycoltetraacetic acid (EGTA), diethylenetriaminepentaacetate (DTPA), hydroxyethylethylenediaminetriacetic acid (HEEDTA), diaminocyclohexanetetraacetic acid (CDRA), 1,2-bis(2-aminophenoxy)ethane-N,N,N′,N′-tetraacetic acid (BAPTA), analogs, derivatives, and prodrugs thereof, and pharmaceutically acceptable salts and hydrates thereof.
